(19) United States
(12) Patent Application Publication
(10) Pub. No.: US 2026/0366002 A1
(43) Pub. Date: Nov. 19, 2026

(54) FUCOSIDASE COMPOSITION AND METHOD FOR DEFUCOSYLATING N-GLYCAN BY USING THE SAME

(71) Applicant: TAIPEI MEDICAL UNIVERSITY, Taipei City (TW)

(72) Inventors: Yves S. Y. HSIEH, Taipei City (TW); Mu-Rong KAO, Taipei City (TW)

(73) Assignee: TAIPEI MEDICAL UNIVERSITY, Taipei City (TW)

(21) Appl. No.: 19/198,703

(22) Filed: May 5, 2025

(51) Int. Cl. C12N 9/44; C07K 16/24; C12N 9/24

(52) U.S. Cl. CPC C12N 9/2451; C07K 16/241; C12N 9/2402; C12Y 302/01051; C12Y 302/01096; C07K 2317/40

(57) ABSTRACT

Provided is an α-L-fucosidase derived from Prevotella nigrescens and a composition including the α-L-fucosidase for defucosylating N-glycans. Also provided is a method for defucosylating N-glycans and producing afucosylated glycoconjugates by using the α-L-fucosidase.

### REFERENCE TO ELECTRONIC SEQUENCE LISTING

The application contains a Sequence Listing which has been submitted electronically in .XML format and is hereby incorporated by reference in its entirety. Said .XML copy, created on May 5, 2025, is named “TPMU-0045US_Sequence Listing.xml” and is 2,252 bytes in size. The sequence listing contained in this .XML file is part of the specification and is hereby incorporated by reference herein in its entirety.

### BACKGROUND

### 1. Technical Field

The present disclosure relates to compositions comprising a fucosidase derived from Prevotella nigrescens, and particularly to methods for producing glycoconjugates having a homogeneous afucosylated glycoform by eliminating core fucoses from N-glycans by using the fucosidase.

### 2. Description of Related Art

Fucosylation is a type of post-translational modification known to modulate the biological functions of glycoproteins and glycolipids. This modification is closely associated with several physiological and pathological processes, including blood group antigen expression, cancer progression, immune response, and antibody-mediated cellular cytotoxicity.

The presence of core L-fucose (L-Fuc) linked to the innermost N-acetylglucosamine (GlcNAc) residue in the N-glycan of the antibody fragment crystallizable (Fc) domain has been shown to reduce the antibody's affinity for FcγRIIIa receptors on immune effector cells such as natural killer (NK) cells and peripheral blood mononuclear cells (PBMCs). This interaction results in diminished antibody-dependent cellular cytotoxicity (ADCC)^{[1]}, which is a key mechanism in the therapeutic function of monoclonal antibodies (mAbs) in targeting malignant or infected cells.

To improve ADCC activity, various strategies have been developed to produce afucosylated antibodies (e.g., trastuzumab (anti-Her2/neu) and rituximab (anti-CD20))^{[2-5]}, including the use of fucose analog inhibitors (e.g., 2-fluoro-L-fucose (2F-Fuc))^{[6,7]} and FUT8 gene knockout Chinese hamster ovary (CHO) cell lines. However, gene knockout approaches may impair the activities of sialyltransferases and other glycosyltransferases, thereby complicating glycan processing^{[8-10]}.

Most therapeutic monoclonal antibodies currently in clinical use exhibit high levels of fucosylation (over 90%), largely due to host cell line characteristics and the need to maintain cell viability. However, reduced fucosylation has been correlated with significantly enhanced ADCC activity. It has also been reported that low fucose IgGs showed at least a 50-fold increase in ADCC activity compared with the fully fucosylated counterpart^{[11-13]}. Thus, effective defucosylation methods are critical to producing therapeutic antibodies with optimized effector functions.

Enzymatic glycoengineering approaches have emerged as practical and efficient solutions for modifying antibody glycosylation. These methods typically involve the sequential enzymatic removal of heterogeneous N-glycans and core fucose residues, followed by transglycosylation to introduce a desired glycan structure at the conserved Fc N-glycosylation site (Asn 297). The removal of heterogeneous N-glycans is carried out using an endo-β-N-acetylglucosaminidase, commonly referred to as “Endo.” Efficient defucosylation relies on α-L-fucosidase enzymes with high specificity and activity toward the core fucose in N-glycans.

Microbial α-L-fucosidases are categorized primarily into glycoside hydrolase families GH29 and GH95. Enzymes in the GH29A subfamily are known for their broad substrate specificity (e.g., acting on α(1,2)-, α(1,3)-, α(1,4)-, and α(1,6)-linked fucosylated substrates), whereas GH29B enzymes tend to exhibit linkage-specific activity (e.g., targeting α(1,3)- and α(1,4)-linked substrates). While a few α-L-fucosidases, such as those derived from Lactobacillus casei and Bacteroides fragilis, have demonstrated activity toward core fucose structures, the availability of enzymes with high efficiency and specificity for α(1,6)-linked fucose remains limited [4,14-16].

Accordingly, there remains a need for improved α-L-fucosidase enzymes that can efficiently and selectively remove core fucoses from antibody Fc glycans, e.g., as part of in vitro glycoengineering processes for the production of afucosylated monoclonal antibodies.

### SUMMARY

In view of the foregoing, the present disclosure provides an α-L-fucosidase derived from Prevotella nigrescens, a composition thereof, and a method for defucosylating N-glycans and thus producing afucosylated glycoconjugates by using the same. The α-L-fucosidase of the present disclosure is highly reactive toward a core glycan motif with fucose α(1,3)-, α(1,4)—, or α(1,6)-linked to GlcNAc, and exhibits excellent thermal and storage stability, retaining its enzymatic activity after prolonged incubation, freezing, and lyophilization over an extended period. Thus, the α-L-fucosidase of the present disclosure is useful for glycoengineering applications.

In at least one embodiment of the present disclosure, an α-L-fucosidase derived from Prevotella nigrescens is provided. The α-L-fucosidase has an amino acid sequence having at least 80% identical to SEQ ID NO: 1. In some embodiments, the amino acid sequence of the α-L-fucosidase may have at least 80% (e.g., 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO: 1, and have the same activity as SEQ ID NO: 1; for example, said amino acid sequence may have the enzymatic activity for hydrolyzing an α(1,6)-linked core fucose present in N-glycans. In some embodiments, the α-L-fucosidase also has an enzymatic activity for hydrolyzing an α(1,3)- or α(1,4)-linked core fucose present in N-glycans.

In at least one embodiment of the present disclosure, the α-L-fucosidase may be a truncated form of SEQ ID NO: 1. In some embodiments, the truncated form of SEQ ID NO: 1 may have amino acids 21 to 452 of SEQ ID NO: 1.

In at least one embodiment of the present disclosure, the α-L-fucosidase may comprise a fusion tag. In some embodiments, the α-L-fucosidase may be a maltose binding protein (MBP)-fusion α-L-fucosidase.

In at least one embodiment of the present disclosure, a composition for defucosylating an N-glycan is provided. The composition comprises the α-L-fucosidase as described above. In some embodiments, the composition of the present disclosure further comprises a reagent that is suitable for fucosidase activity. In some embodiments, the reagent comprised in the composition includes, but is not limited to, water, sodium phosphate buffer, sodium acetate, or 2-(N-morpholino) ethanesulfonic acid (MES).

In at least one embodiment of the present disclosure, the composition excludes a divalent cation selected from the group consisting of Cu²⁺ and Zn²⁺.

In at least one embodiment of the present disclosure, the composition has a pH value in a range from 4.0 to 7.0. In some embodiments, the composition has a pH value of about 4.0, about 4.5, about 5.0, about 5.5, about 6.0, about 6.5, or about 7.0.

In at least one embodiment of the present disclosure, the α-L-fucosidase is present in a lyophilized form. In some embodiments, the composition is present in a lyophilized form.

In at least one embodiment of the present disclosure, the composition further comprises a glycosidase or a glycosynthase. In some embodiments, the glycosidase may be an endoglycosidase, an exoglycosidase, or a combination thereof. In some embodiments, the glycosidase is selected from the group consisting of endo-β-N-acetylglucosaminidase, galactosidase, neuraminidase, mannosidase, and any combination thereof. In some embodiments, the glycosidase or the glycosynthase is selected from the group consisting of EndoA, EndoCC, EndoF1, EndoF2, EndoF3, EndoH, EndoM, EndoS, EndoS2, and a variant thereof. In some embodiments, the glycosynthase may be a variant of the endoglycosidase, such as a mutant of endo-β-N-acetylglucosaminidase (e.g., Endo-S2 mutant D184M).

In at least one embodiment of the present disclosure, a method for defucosylating an N-glycan is provided. The method for defucosylating the N-glycan comprises providing the α-L-fucosidase as described above and a glycoconjugate including the N-glycan having an α(1,6)-linked core fucose; and incubating the α-L-fucosidase with the glycoconjugate under a condition for cleaving the α(1,6)-linked core fucose from the N-glycan.

In at least one embodiment of the present disclosure, the method for defucosylating the N-glycan further comprises incubating the glycoconjugate with an endo-β-N-acetylglucosaminidase and deglycosylating the glycoconjugate to produce a GlcNAc acceptor that retains the α(1,6)-linked core fucose. In some embodiments, the method for defucosylating the N-glycan further comprises cleaving the α(1,6)-linked core fucose from the GlcNAc acceptor by the α-L-fucosidase. In some embodiments, the incubation of the α-L-fucosidase with the glycoconjugate comprises cleaving the α(1,6)-linked core fucose retained on the GlcNAc acceptor after the deglycosylation.

In at least one embodiment of the present disclosure, the glycoconjugate is incubated simultaneously with the α-L-fucosidase and the endo-β-N-acetylglucosaminidase. In some embodiments, the cleavage of the α(1,6)-linked core fucose and the deglycosylation of the glycoconjugate occur simultaneously in a single reaction mixture.

In at least one embodiment of the present disclosure, the incubation of the α-L-fucosidase with the glycoconjugate is performed at about 35 °C to about 65 °C. In some embodiments, the incubation is performed at about 35 °C, about 40 °C, about 45 °C, about 50 °C, about 55 °C, about 60 °C, or about 65 °C.

In at least one embodiment of the present disclosure, the incubation of the α-L-fucosidase with the glycoconjugate is performed at a pH value in a range from 4.0 to 7.0. In some embodiments, the incubation is performed at a pH value of about 4.0, about 4.5, about 5.0, about 5.5, about 6.0, about 6.5, or about 7.0.

In at least one embodiment of the present disclosure, a method of producing an afucosylated glycoconjugate is provided. The method of producing the afucosylated glycoconjugate comprising: deglycosylating a glycoconjugate to produce a GlcNAc acceptor; removing an α(1,6)-linked core fucose by exposing the GlcNAc acceptor to the α-L-fucosidase as described above under a condition for defucosylating the GlcNAc-acceptor to obtain an afucosylated GlcNAc acceptor; and enzymatically reacting the afucosylated GlcNAc acceptor with a sugar donor to produce the afucosylated glycoconjugate. In some embodiments, the sugar donor carries a predetermined number and type of sugar residues. In some embodiments, the sugar donor includes, but is not limited to, a synthetic oligosaccharide oxazoline and a sialylated oxazoline.

In at least one embodiment of the present disclosure, the deglycosylation is carried out by endo-β-N-acetylglucosaminidase or a variant thereof.

In at least one embodiment of the present disclosure, the enzymatical reaction of the afucosylated GlcNAc acceptor with the sugar donor is carried out by an endoglycosidase variant.

In at least one embodiment of the present disclosure, the method of producing the afucosylated glycoconjugate is performed in a one-pot manner.

In at least one embodiment of the present disclosure, the glycoconjugate to which the α-L-fucosidase of the present disclosure is applied includes, but is not limited to, a glycolipid, a glycoprotein, an oligosaccharide, and a glycopeptide. In some embodiments, the glycoconjugate is a therapeutic glycoprotein. In some embodiments, the glycoconjugate may be selected from the group consisting of erythropoietin, a blood clotting factor, influenza hemagglutinin, an interferon, a colony-stimulating factor, a cytokine, an antibody, a Fc-fusion protein, an enzyme, and a hormone. In some embodiments, the glycoprotein is a monoclonal antibody.

As mentioned above, the present disclosure provides a novel fucosidase derived from Prevotella nigrescens and a method for obtaining a glycosylated protein having increased levels of afucosylated glycoforms. Therefore, the fucosidase and the method provided herein are useful in glycoengineering processes for the production of afucosylated monoclonal antibodies with improved therapeutic outcomes.

### BRIEF DESCRIPTION OF THE DRAWINGS

The present disclosure can be more fully understood by reading the following detailed descriptions of the embodiments, with reference made to the accompanying drawings.

FIG. 1 shows the phylogenic analysis of glycoside hydrolase family 29 (GH29) α-L-fucosidases.

FIGS. 2A to 2E show the identification of potential nucleophile and general acid/base residues in PnfucA through protein structure alignment. FIG. 2A shows the amino acid residues with the confidence value or predicted Local Distance Difference Test (pLDDT) lower than 50 (colored in red) or higher than 90 (colored in blue), respectively. FIG. 2B shows the predicted structure of PnfucA exhibiting a (β/α)₈ barrel with an N-terminal β-sandwich domain. The α-helix, β-sheet, and loop regions are colored in red, yellow, and green, respectively. FIGS. 2C to 2E show the structure superposition between PnfucA and Thermotoga maritina TM aFuc (PDB: 1HL9, FIG. 2C), Homo sapien FucA1 (PDB: 7PLS, FIG. 2D), and Lactobacillus casei AlfC (PDB: 6OHE, FIG. 2E). PnfucA is in color, while the other α-L-fucosidases are in grey. The catalytic residues are represented by sticks.

FIG. 3 shows sodium dodecyl sulfate polyacrylamide gel electrophoresis (SDS-PAGE) analysis of partially purified recombinant α-L-fucosidases AlfC and PnfucA, and purified Δ20PnfucA. The α-L-fucosidases AlfC from Lactobacillus casei and PnfucA from Pretonella nigrescens, expressed with an N-terminal maltose binding protein (MBP)-fusion tag, are indicated with an arrow.

FIG. 4 shows the kinetic study of recombinant α-L-fucosidases PnfucA and AlfC, in which the enzyme reactions are performed using p-nitrophenyl-α-L-fucopyranoside (pNP-Fuc) at different concentrations in 50 mM sodium phosphate buffer (pH 6.5).

FIGS. 5A to 5F show the effects of temperature, pH, and divalent ions on the catalytic activity of Δ20PnfucA. FIG. 5A demonstrates the optimal reaction temperature, as determined by incubating the enzyme at different temperatures. FIG. 5B demonstrates the thermostability of the enzyme, as determined by incubating the enzyme at different temperatures for 10 min before performing the enzymatic activity assay. FIG. 5C demonstrates the optimal pH, as determined using sodium acetate (pH 4 to pH 5.5, circle), sodium phosphate (pH 6 to pH 7.5, square), and Tris (pH 8 to 9.5, triangle) buffers. FIG. 5D demonstrates the effects of divalent ions on enzyme activity, as determined using 50 mM sodium phosphate buffer (pH 7) containing 5 mM of either divalent ions or ethylenediaminetetraacetic acid (EDTA). FIG. 5E demonstrates the effects of incubation time, as determined by incubating the enzyme at 37 °C in sodium phosphate buffer (pH 7) for up to 6 hours. FIG. 5F demonstrates the effects of storing the enzyme in 50 mM sodium phosphate buffer at 4 °C or −20 °C, or as a freeze-dried powder at 4 °C, after storage for 28 days.

FIG. 6 shows the substrate specificity of Δ20PnfucA. Dark grey circle: glucose; Dark grey square: N-acetylglucosamine; Light grey circle: galactose; Light grey square: N-acetylgalactosamine; Triangle: fucose; Diamond: sialic acid; Circle with stripes: mannose. Hydrolysis rate is determined by high-performance liquid chromatography (HPLC) analysis, and the values indicate the mean±standard deviation. Hydrolysis rate is determined by liquid chromatography-mass spectrometry (LC-MS) analysis.

FIG. 7 shows the composition, mass, and retention time of N-glycosylated peptides from thermolysin-digested adalimumab, detected by the LC-MS method. Abbreviations: mono-N-acetylglucosaminylated (G0-N), agalactosylated (G0), mono-galactosylated (G1), mono-sialylated (G2-NANA), and di-sialylated (G2+2NANA) complex-type N-glycans. F: with core-fucosylation. Dark grey square: N-acetylglucosamine; Light grey circle: galactose; Triangle: fucose; Diamond: sialic acid; Circle with stripes: mannose.

FIGS. 8A and 8B show the removal of the core α(1-6) fucose of adalimumab by Δ20PnfucA, in which the hydrolysis products are loaded for SDS-PAGE (FIG. 8A) and for lectin blotting (FIG. 8B). Lane 1: untreated adalimumab; Lane 2: Endo-S2 deglycosylation; Lane 3: Δ20PnfucA defucosylation; Lane 4: combined Endo-S2 and Δ20PnfucA treatment; Lane 5: PNGase F deglycosylation; Lane 6: fetuin (afucosylated N-linked glycoprotein; Lane 7: bovine serum albumin (BSA). Endo-S2 is indicated by “*”.

FIG. 9 shows the defucosylation of adalimumab using a combination of wild-type Endo-S2 and an α-L-fucosidase, in which adalimumab (50 μg) is treated with Endo-S2 (5 μg) combined with either Δ20PnfucA (5 μg) or MBP-AlfC (5 μg), and 2 μg of protein is loaded for lectin blotting analysis. Samples are collected at 0 hours, 2 hours, 4 hours, and 6 hours post hydrolysis.

FIG. 10 shows multiple sequence alignment of α-L-fucosidases, in which the identical amino acid residues among the four enzymes are highlighted, and the catalytic nucleophilic and general acid-base residues are indicated in italic and underlined bold text, respectively. H. sapiens: Homo sapiens α-L-fucosidase FucA1 (NP 000138.2); T. maritima: Thermotoga maritima α-L-fucosidase TM aFuc (AAD35394.1); L. casei: Lactobacillus casei α-L-fucosidase AlfC (CAQ67984.1); P. nigrescens: Prevotella nigrescens α-L-fucosidase PnfucA (PDB: 7PLS).

FIG. 11 shows the representation of the Fucα(1,6) GlcNAc binding pocket in Lactobacillus casei AlfC, in which the amino acids involved in substrate binding are represented in sticks. AlfC and PnfucA are colored in grey and green, respectively.

FIGS. 12A to 12D show the transglycosylation of afucosylated adalimumab with sequential deglycosylation/transglycosylation reactions, or with a One-Pot Two-step reaction, in which the post-hydrolysis and transglycosylation samples are analyzed using SDA-PAGE (FIG. 12A) and lectin blotting (FIG. 12B). Lane 1: untreated adalimumab (Hum_{Ori}); Lane 2: combined wild-type Endo-S2 (S2WT) and Δ20PnfucA (fucA) treatment; Lane 3: transglycosylation of afucosylated adalimumab (Hum_{GlcNAc}) with Endo-S2 mutant D184M (S2_{Mut}) and SCT-oxazoline (SCT-ox); Lane 4: combined Endo-S2 mutant D184M (S2_{Mut}) and Δ20PnfucA (fucA) treatment; Lane 5: transglycosylation after the 3 hour-treatment with the Endo-S2 mutant D184M and Δ20PnfucA, followed by the immediate addition of SCT N-glycan oxazoline (+SCT-ox). FIG. 12C demonstrates the composition of the Fc N-glycans. Dark grey square: N-acetylglucosamine; Light grey circle: galactose; Triangle: fucose; Diamond: sialic acid; Circle with stripes: mannose. FIG. 12D demonstrates the SDS-PAGE analysis of purified adalimumab (Hum_{Ori}) and deglycosylated/afucosylated adalimumab (Hum_{GlcNAc}).

FIG. 13 shows the monitoring of transglycosylation products (Hum_{SCT}) in the presence of different SCT-ox concentrations.

FIG. 14 shows the ELISA dose-response curve for glycoengineered adalimumab with the Fcγ receptor IIIa, in which the adalimumab glycoforms, Humor, Hum_{SCT}, and Hum_{GlcNAc} are obtained from the One-Pot Two-Step reaction and purified by Protein A chromatography.

### DETAILED DESCRIPTION OF THE EMBODIMENTS

The description discloses some embodiments in such a detail that a person skilled in the art is able to utilize the embodiments based on the disclosure. Not all steps or features of the embodiments are discussed in detail, as many of the steps or features will be obvious to a person skilled in the art based on this disclosure.

It is further noted that, as used in this disclosure, the singular forms “a,” “an,” and “the” include plural referents unless expressly and unequivocally limited to one referent. The term “or” is used interchangeably with the term “and/or” unless the context clearly indicates otherwise.

As used herein, the term “about” generally means within 10%, 5%, 1%, or 0.5% of a given value or range. Alternatively, the term “about” means within an acceptable standard error of the mean when considered by one of ordinary skill in the art. Unless otherwise expressly specified, all of the numerical ranges, amounts, values, and percentages, such as those for quantities of materials, durations of time periods, temperatures, operating conditions, ratios of amounts, and the likes disclosed herein, should be understood as modified in all instances by the term “about.”

As used herein, the term “comprising” or “comprises” is used in reference to compositions, methods, and respective component(s) thereof, which are included in the present disclosure, yet open to the inclusion of unspecified elements.

The present disclosure provides an α-L-fucosidase derived from Prevotella nigrescens, which has an amino acid sequence having at least 80% identical to SEQ ID NO: 1. The present disclosure also provides a composition for defucosylating an N-glycan, which comprises the α-L-fucosidase and a method for defucosylating an N-glycan and producing an afucosylated glycoconjugate by using said α-L-fucosidase.

As used herein, the term “sequence identity” means the percentage that the amino acid or nucleotide residues of a candidate protein or nucleic acid fragment are completely identical to the amino acid or nucleotide residue of a reference protein or nucleic acid fragment. When performing the above comparison, said candidate protein or nucleic acid fragment may be aligned, and the gaps may be introduced as necessary, so as to form the highest sequence identity between the two sequences. The amino acid residue where is conservative substitution is regarded as different residue, and the nucleotide residues which are degenerated codons are also regarded as different residues, when calculating the identity; for example, it is considered that there is a different residue U or C between the codons AAU and AAC that both encode asparagine.

It should be understood that compared to the amino acid or nucleotide sequence of a reference protein or nucleic acid fragment in the present disclosure, amino acid or nucleotide sequence of a candidate protein or nucleic acid fragment, which is modified (e.g., deleted, substituted, or added) at least a part in the sequence, is also within the scope of the present disclosure, as long as the resulting candidate protein or nucleic acid fragment has substantially the same biological activity as the amino acid or nucleotide of the reference protein or nucleic acid fragment. This results from the codon degeneracy and the functional tolerance of certain structural variations. Moreover, substantially identical or homologous polypeptides include additions, truncations, internal deletions or insertions, conservative and non-conservative substitutions, or other modifications located at positions of the amino acid sequence which do not destroy the function of the protein. In other words, a person having ordinary skill in the art can understand that the amino acid sequence of the α-L-fucosidase may have variations, as long as the structure of the active region and the important amino acids in the amino acid sequence of the α-L-fucosidase are not changed, and the effect of defucosylation can be achieved.

As used herein, the term “N-glycan” refers to a polymer of sugars that has been released from a glycoconjugate but was formerly linked to the glycoconjugate via a nitrogen linkage.

According to the embodiments of the present disclosure, the α-L-fucosidase may be applied to a glycoconjugate for defucosylation.

As used herein, the term “glycoconjugate” encompasses all molecules in which at least one sugar moiety is covalently linked to at least one other moiety. The term specifically encompasses all biomolecules with covalently attached sugar moieties, such as glycolipids, glycoproteins, oligosaccharides, glycopeptides, and proteoglycans.

As used herein, the term “glycolipid” refers to a lipid molecule having one or more covalently attached sugar moieties (i.e., glycans), which may include monosaccharides, disaccharides, oligosaccharides, and/or polysaccharides. The glycan moiety may be linear or branched and, in certain embodiments, may further comprise functional groups such as sulfate and/or phosphate groups.

As used herein, the term “glycoprotein” refers to a molecule comprising one or more polypeptides or proteins covalently linked to one or more oligosaccharide chains (e.g., glycans). The glycan may be O-linked or N-linked.

According to the embodiments of the present disclosure, the glycoconjugate may be a therapeutic glycoprotein. Representative therapeutic glycoproteins include, but are not limited to, erythropoietin, a blood clotting factor, influenza hemagglutinin, an interferon, a colony-stimulating factor, a cytokine, an antibody, a Fc-fusion protein, an enzyme, and a hormone.

As used herein, the term “antibody” or “immunoglobulin molecule” refers to a molecule that contains an antigen binding site which specifically binds an antigen or an Fc region that binds to cell receptors. Structurally, the simplest naturally occurring antibody (e.g., IgG) comprises four polypeptide chains, two heavy (H) chains and two light (L) chains inter-connected by disulfide bonds. The natural immunoglobulins represent a large family of molecules that include several types of molecules, such as IgD, IgG, IgA, IgM, and IgE. The term also encompasses hybrid antibodies, or altered antibodies, and fragments thereof, including Fc fragment(s).

In at least one embodiment of the present disclosure, the glycoconjugate may be an antibody, including but not limited to therapeutic monoclonal antibodies used for the treatment of diseases, such as cancers, autoimmune diseases, inflammatory disorders, cardiovascular diseases, infectious diseases, and transplant rejection. Exemplary antibodies include 17b, 48d, A32, C11, 2G12, F240, IgG1b12, 19e, X5, TNX-355, cetuximab, rituximab, muromonab-CD3, abciximab, daclizumab, basiliximab, palivizumab, infliximab, adalimumab, certolizumab pegol, trastuzumab, pertuzumab, gemtuzumab ozogamicin, alemtuzumab, ibritumomab tiuxetan, tositumomab, omalizumab, efalizumab, bevacizumab, ranibizumab, panitumumab, I-131 tositumomab, natalizumab, etanercept, IGN101, volociximab, anti-CD80 mAb, anti-CD23 mAb, CAT-3888, CDP-791, MDX-010, MDX-060, MDX-070, matuzumab, CP-675206, CAL, SGN-30, zanolimumab, adecatumumab, oregovomab, nimotuzumab, ABT-874, denosumab, AM 108, AMG 714, fontolizumab, golimumab, CNTO 1275, ocrelizumab, HuMax-CD20, belimumab, epratuzumab, MLN1202, visilizumab, tocilizumab, eculizumab, pexelizumab, mepolizumab, and MYO-029.

Many examples have been used to illustrate the present disclosure. The examples below should not be taken as a limit to the scope of the disclosure.

### EXAMPLES

### Materials and Methods

The materials and methods used in the following Examples 1 to 6 are described in detail below. The materials used in the present disclosure but unannotated herein are commercially available.

### (1) Chemicals and Plasmids

Analytical or reagent-grade chemicals were used unless specified and were from Fisher Scientific (UK) and Sigma-Aldrich (St. Louis, US). The synthesized and codon-optimized DNA sequences encoding the α-L-fucosidases of Prevotella nigrescens (PnfucA) and Lactobacillus casei (AlfC), as well as the expression vectors (pET-16b, pET-22b+ and pMAL-c4X) were from GenScript (US).

### (2) Evolutionary Study of α-L-Fucosidases

Protein sequences of α-L-fucosidases were selected from databases including the National Center for Biotechnology Information (NCBI), Carbohydrate-Active enZYmes (CAZy), and the RCSB Protein Data Bank (PDB). Multiple sequence alignment was conducted with Clustal Omega, and the phylogenetic tree was built using the Neighbor-Joining method with MEGA11. Signal peptides were predicted with SignalP version 6.0. The 3D structure of PnfucA was predicted using AlphaFold 2 and visualized with the PyMol Molecular Graphics System (Version 1.2r3pre, Schrodinger, LLC).

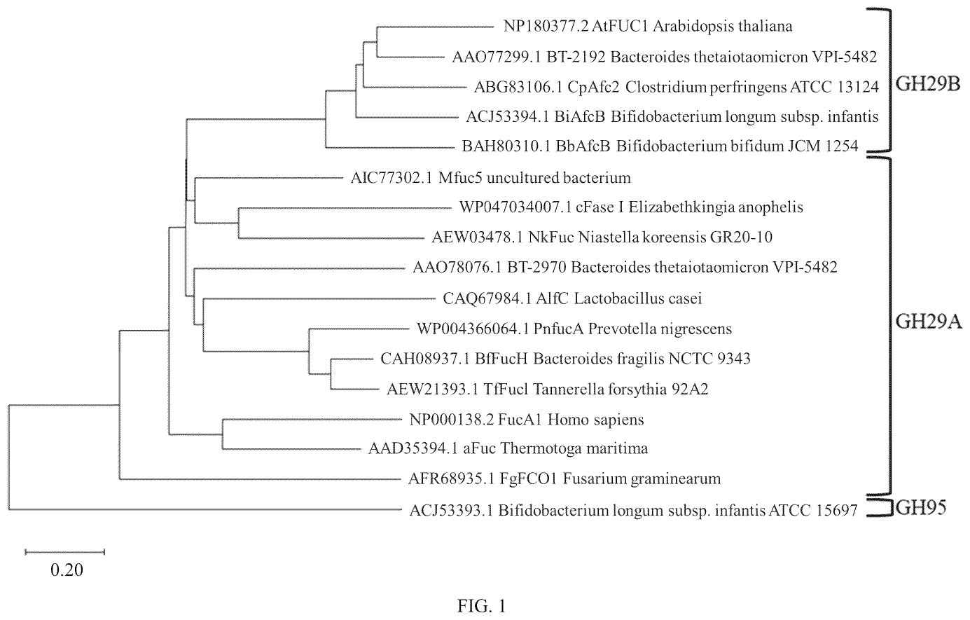

The α-L-fucosidases analyzed herein and their corresponding sequence accession numbers are detailed in FIG. 1.

### (3) Expression and Purification of Recombinant α-L-Fucosidases

The coding sequence of α-L-fucosidase was amplified using polymerase chain reaction (PCR) with the Phusion High-Fidelity DNA Polymerase (Thermo Fisher Scientific Baltics, Lithuania), following the manufacturer's recommendations. It was then cloned into the expression vector after linearization with appropriate restriction enzymes, which were purchased from New England BioLabs (NEB, UK). The resulting expression vectors were transformed into Escherichia coli (E. coli) DH5a strain (Yeastern Biotech, Taiwan) for plasmid preservation and subsequently into the E. coli BL21 strain (Yeastern Biotech, Taiwan) for recombinant protein expression. Transformants were selected from Luria-Bertani (LB) medium agar plates supplemented with 100 μg/mL ampicillin.

For recombinant enzyme production, a single bacterial colony was inoculated into LB medium supplemented with ampicillin and cultured at 37 °C overnight. The next day, fresh LB medium was inoculated with the overnight culture to obtain a starting optical density (OD₆₀₀) of 0.1 and was cultured at 37 °C until the OD₆₀₀ reached between 0.6 and 0.8. The culture was then induced with 0.5 mM isopropyl 1-thio-β-D-galactopyranoside (IPTG, AK Scientific, US), and incubation continued at 16 °C for an additional 16 hours. Cells were collected by centrifugation at 6000 g for 5 min. The resulting cell pellet was resuspended in binding buffer for protein purification, and cell lysis was performed with the One Shot Cell Disruptor equipment (Constant Systems, UK).

The purification of recombinant α-L-fucosidases was carried out by affinity chromatography with an AKTA Purifier FPLC System (GE Healthcare, Sweden). His-tagged recombinant enzymes were purified with a HisTrap FF column (Cytiva, Sweden) and a phosphate binding buffer (20 mM sodium phosphate, 500 mM NaCl, and 20 mM imidazole, pH 7.4). After the sample injection, the column was washed with 15 column volumes (CV) of binding buffer and subsequently eluted with 20 CV of elution buffer (20 mM sodium phosphate, 500 mM NaCl, and 500 mM imidazole, pH 7.4) with a linear gradient from 0 to 100%. For maltose-binding protein (MBP)-fusion proteins, purification was performed with an MBPTrap HP column (Cytiva, Sweden). The binding buffer contained 20 mM Tris-HCl, 200 mM NaCl, and 1 mM ethylenediaminetetraacetic acid (EDTA) at pH 7.4, and the elution buffer was prepared with the same buffer supplemented with 10 mM maltose.

The purity of proteins in the elution fractions was verified by sodium dodecyl-sulfate polyacrylamide gel electrophoresis (SDS-PAGE) analysis using the TGX Stain-Free FastCast Acrylamide Kit (Bio-Rad, US). Fractions showing a strong band of the target protein were gathered and concentrated using an Amicon Ultra Centrifugal Filter Unit (Merck, Germany). Following buffer exchange with 50 mM sodium phosphate buffer (pH 6.5), the protein concentration of the purified enzyme was determined by the Bradford assay^{[17]} using Bio-Rad Protein Assay (Bio-Rad, US), with bovine serum albumin (BSA) serving as the standard.

### (4) Enzymatic Activity Experiment

The enzyme activity of α-L-fucosidases was determined with 0.03 μg of purified Δ20PnfucA and 2 mM of the artificial substrate p-nitrophenyl-α-L-fucopyranoside (pNP-fucose, Carbosynth, UK). The reaction was carried out in 50 mM sodium phosphate buffer (pH 6.5) for 5 min at 37 °C. The hydrolysis product, pNP, was detected by spectrophotometry using a BioDrop Duo+ (Biochrom, UK) at OD₄₀₅. Its concentration was quantified using a standard curve generated from serially diluted pNP solutions, ranging from 0.016 mM to 0.5 mM. One enzyme unit (U) was defined as 1 μmol of pNP released per minute.

The effects of pH were determined using 50 mM sodium acetate (pH 4 to 5.5), sodium phosphate (pH 6 to 7.5), and Tris (pH 8 to 9.5) buffers. The effects of temperature were determined across a range of temperatures from 20 °C to 70 °C. Thermostability was determined by incubating 0.045 μg of purified enzyme at temperatures ranging from 25 °C to 70 °C before testing the enzymatic activity. Relative activity was determined in comparison with the highest activity value measured among different temperatures or pH.

The effects of divalent ions were determined in 50 mM sodium phosphate buffer (pH 7) containing 5 mM various divalent ions (Mg²⁺, Co²⁺, Ni²⁺, Cu²⁺, Zn²⁺, or Ca²⁺) or EDTA. Relative activity was determined in comparison with the enzyme activity measured in the sample without addition of divalent ions (i.e., buffer only).

The effect of incubation time was determined by incubating the purified Δ20PnfucA in 50 mM sodium phosphate buffer (pH 7) up to 6 hours. Relative activity was determined in comparison with the time point zero (i.e., t=0 h).

To examine the enzyme's stability, the purified Δ20PnfucA, maintained at a concentration of 2 mg/mL in sodium phosphate buffer (pH 7), underwent various storage conditions: 1) stored at 4 °C for 28 days; 2) stored at −20 °C for 28 days, followed by direct enzyme assay after thawing; 3) underwent freeze-drying before being stored at 4 °C for 28 days. Relative activity was determined in comparison with the enzyme activity measured with freshly purified enzyme on day 1.

The kinetics study was conducted with pNP-fucose concentrations ranging from 0.0625 mM to 16 mM. The Kₘ and Vₘₐₓ values were determined through nonlinear curve fitting utilizing the Michaelis-Menton model in Prism 10 (GraphPad, US).

### (5) Substrate Specificity Experiment

The fucosylated oligosaccharide substrates were synthesized as previously described^{[18,19]}. The reaction was performed in a final volume of 50 μL, containing 1 μg of purified Δ20PnfucA and 2 mM of the fucosylated substrate with a C-5 amino linker (Formula I), in 50 mM sodium phosphate buffer (pH 7), and incubated at 37 °C for 16 hours.

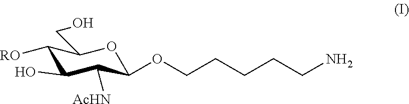

After the hydrolysis, the reaction products were purified using a Bond Elut Carbon cartridge (Agilent, US) that contained 50 mg of carbon in a 1-mL column. The carbon was activated with 3 mL of 80% acetonitrile and then washed with 3 mL of MilliQ (MQ) water. A 200 μL aliquot diluted with MQ water was injected onto the column, which was then washed with 3 mL of MQ water and eluted with 500 μL of 50% acetonitrile. The eluted fraction was freeze-dried overnight.

The hydrolysis products of the fucosylated substrates were analyzed by high performance liquid chromatography (HPLC) and mass spectrometry (MS) methods. Before HPLC analysis, dried samples were dissolved in 50 mM sodium phosphate buffer (pH 8) (19 μL), labeled with 1 μL of 12 mM Cy5 mono N-hydroxysuccinimide (NHS) ester (GE Amersham, US), and incubated at room temperature overnight. Analytical HPLC was carried out with an XBridge Glycan Ethylene Bridged Hybrid (BEH) amide Column (130 Å, 3.5 μm, 2.1 mm×150 mm, Waters) on a Waters e2695 Separation Module, paired with a Waters 2998 photo diode array (PDA) detector. Cy5-labeled glycans were detected with an absorption wavelength at 649 nm. The mobile phase consisted of solvent A (100 mM ammonium formate, pH 4.5) and solvent B (100% acetonitrile), and the separation was conducted with a gradient elution from 90 to 45% of solvent B over 40 min (flow rate, 0.25 mL/min). High resolution MS was performed on an LTQ Orbitrap XL ETD (Electron Transfer Dissociation) mass spectrometer (Thermo Fisher Scientific, US) equipped for ultra performance liquid chromatography (UPLC; Waters Acquity) with an XBridge BEH C18 Column (130 Å, 3.5 μm, 1 mm×150 mm, Waters).

### (6) Protein Deglycosylation Experiment

The deglycosylation by Streptococcus pyogenes endo-β-N-acetylglucosaminidase (Endo-S2) was performed with 50 μg protein substrate and 5 μg Endo-S2 wild-type or mutant D184M enzymes in 50 mM sodium phosphate buffer (pH 7) at 37 °C for 3 hours. The complete removal of N-glycans was performed with 10 μg protein substrate and the peptide-N-glycosidase F (PNGase F, NEB) according to the manufacturer's recommendation. Fetuin from fetal bovine serum and BSA served as control proteins.

### (7) Endo-S2 Mutant D184M Glycosynthase Activity Experiment

To evaluate the glycosynthase efficiency of Endo-S2 mutant D184M, the hydrolysis reaction was monitored by incubating 100 μg original adalimumab (Hum_{Ori}) with 1 μg purified Endo-S2 mutant in a Tris buffer (50 mM Tris-HCl, 50 mM NaCl, and 1 mM CaCl₂), pH 7.6) at 37 °C for 1 hour, while the transglycosylation rate was determined by incubating 100 μg deglycosylated adalimumab with 1 μg purified Endo-S2 mutant in the presence of 10 mM sialyl-complex-type N-glycan-oxazoline (SCT-ox) under the same reaction conditions. To evaluate the SCT-ox concentration effects on the transglycosylation efficiency, 100 μg Hum_{Ori} was treated with 10 μg Endo-S2 mutant D184M and 10 μg Δ20PnfucA in 50 mM Tris buffer (supplemented with 50 mM NaCl and 1 mM CaCl₂), pH 7.6) at 37 °C for 3 hours. Then, the transglycosylation reaction was initiated by adding 0.1 mM, 1 mM, or 10 mM SCT-ox in the reaction mixture, and the incubation continued at 37 °C for 6 hours. The reaction products were taken after 1, 2, 4, and 6 hours, purified by Protein A chromatography, and then digested by thermolysin treatment. The N-glycan composition was determined by LC-ESI-MS analysis.

### (8) Deglycosylation and Transglycosylation of Adalimumab (Humira)

The deglycosylation reaction was performed with 50 μg adalimumab and 5 μg Endo-S2 in 50 mM sodium phosphate buffer (pH 7) at 37 °C for 3 hours. The defucosylation reaction was performed with 50 μg Endo-S2-treated adalimumab and 5 μg α-L-fucosidase in 50 mM sodium phosphate buffer (pH 7) at 37 °C for 3 hours.

Transglycosylation was conducted at 37 °C for 1 hour with 50 μg of deglycosylated protein acceptor, 5 μg of Endo-S2 D184M mutant, and 10 mM sialyl complex-type N-glycan oxazoline (SCT-ox, produced in-house) in 50 mM Tris-HCl buffer (pH 7.6), complemented with 50 mM NaCl and 1 mM CaCl₂.

Enzyme-treated adalimumab was purified using the Protein A Spin Antibody Purification Kit (BioVision, US). The purified adalimumab was then digested by thermolysin, and the Fc domain N-glycan was determined using LC-ESI-MS on an Orbitrap Fusion MS (Thermo Scientific, US) equipped with an Easy-nLC 1200 system and an Easy-Spray source. Samples were injected into a C18 Easy column (0.075 mm×150 mm, ID 3 μm).

### (9) Lectin Blotting

The presence of α(1,6)-linked core-fucose in glycoproteins was detected using Western blotting with biotinylated Aleuria aurantia lectin (AAL) (Vector Laboratories, US). After electrophoretic separation, sample proteins were transferred onto the Immun-Blot PVDF membrane (Bio-Rad, US) using the Criterion blotter system (Bio-Rad, US). The membrane was then incubated at 4 °C overnight in a blocking solution composed of PBS (OmicsBio, Taiwan) supplemented with 0.05% Tween-20 (PBS-T) and 3% BSA. The blotting process started with an incubation in 10 mL of blocking buffer containing 2 μg/mL AAL at room temperature for 1 hour, followed by washing three times for 30 min each with PBS-T. Subsequently, the membrane was incubated in 10 mL of blocking buffer supplemented with 10 μL of Avidin-horseradish peroxidase (HRP) conjugate (Invitrogen, US) at room temperature for 1 hour. After washing, the fucosylated N-linked glycans were visualized upon the addition of Western Lightning Plus Chemiluminescent Substrate (PerkinElmer, US), and images were captured using the iBright 1500 Imaging System (Thermo Scientific, US).

### (10) Determination of Glycan-Engineered Adalimumab Binding to FcγRIIIa

The binding activity was determined by ELISA assay. The FcγRIIIa V158 (GST-Fusion, AB Bioscience) (0.5 μg/mL) was coated on a 96F MaxiSorp ELISA plate (NUNC) at 4 °C overnight in 100 μL of coating buffer composed of 50 mM carbonate-bicarbonate (pH 9.6). Then, the plate was washed three times with a washing buffer composed of PBS with 0.05% Tween 20 (PBS-T), and incubated at room temperature for 2 hours after the addition of 200 μL of blocking buffer (2% BSA in PBS buffer). After the blocking step, the plate was washed with PBS-T before adding 200 μL of original adalimumab (Hum_{Ori}), deglycosylated and afucosylated adlimumab (Hum_{GlcNAc}), and homogeneous and afucosylated SCT-glycan adalimumab (Hum_{SCT}). Those samples were serially diluted in assay buffer (1% BSA in PBS) at concentrations ranging from 4,000 ng/mL to 0.49 ng/mL, and the plate was incubated at 4 °C overnight. On the third day, after washing the plate three times with PBS-T, 100 μL of Goat (Fab′)₂ anti-human IgG Fcγ-HRP (Jackson ImmunoResearch Laboratories) diluted 1:10000 in assay buffer were added per well, and the plate was incubated at room temperature for 1 hour. After the last washing step, 100 μL of 3,3′,5,5′-tetramethylbenzidine (TMB) ELISA substrate (Abcam) were added. After at least 5 min in the dark, the color formation was stopped with 100 μL of stop solution (2 N H₂SO₄), and the ELISA plate was read with a spectrophotometer (Varioskan Lux, Thermo Scientific) for the absorbance at OD₄₅₀. The half maximal effective concentration (EC50) values were determined through nonlinear curve fitting utilizing the Sigmoidal 4PL model in Prism 10 (GraphPad, US).

### Example 1: Bioinformatic Analysis of the PnfucA Sequence

The phylogenetic tree was constructed using the Neighbor-Joining method in MEGA 11. As shown in FIG. 1, fucosidases that exhibit linkage selectivity towards α(1,3)- and α(1,4)-linked substrates were classified in the GH29B subgroup, while those with broad substrate specificity were classified in the GH29A subgroup. The bacterial GH95 α-L-fucosidase from Bifidobacterium longum strain ATCC 15697 was used as the outgroup.

PnfucA belongs to the GH29A subfamily, but it exhibited relatively low sequence identity when compared with other characterized GH29A α-L-fucosidases (FIG. 1). The amino acid sequence of PnfucA is as follows: “MKRIITTSLL GIALAIPIFA QTNSQQQESN NRATTTESVP QKYIPTAENL AARKRFEGFR FGIFLHWGIY STFAQGEWYL NNKINKDEYA KAASAFYPSY FDADAWISAI KASGAKYITF TSRHHDSFSM FKTKESNYNI VDATPFKRDV LKELANACHK QNIDLHIYYS ILDWIREDYP IGRTGLYTGR KLKPNYGTYF NEMKGQVSEL LHNYGKVGAI WLDGYWDHDS DSIPFDWRME EFYRYIHSIQ PACLIGNNHH ITPIDGEDFQ MFERDLPGEN KAGLSGQAIS RLPLEACQTM NGMWGYKVSD INYKSTSQLI ELLVRSAAKG SNLLLNIGPQ PNGELPALAL DRLQGIGKWM QQYGETIYET MAGDVAEADW GVSTSKGKKT YLHVLEQDAK TISCALSVKP KRVIDFASRQ PLSFHYDKKT KTLTTHIGSH RNVVDYIIEI EK” (SEQ ID NO. 1).

Specifically, PnfucA shared only 18.6% sequence identity with Lactobacillus casei α-L-fucosidases (AlfC), 21.7% with Thermotoga maritina α-L-fucosidases (TM aFuc), and 53.1% identity to the closest α-L-fucosidase homolog, the Bacteroides fragilis BfFucH. The conserved catalytic nucleophile D223 in PnfucA had been putatively identified, and was aligned to both nucleophiles D224 in TM aFuc and D200 in AlfC^{[20,21]}.

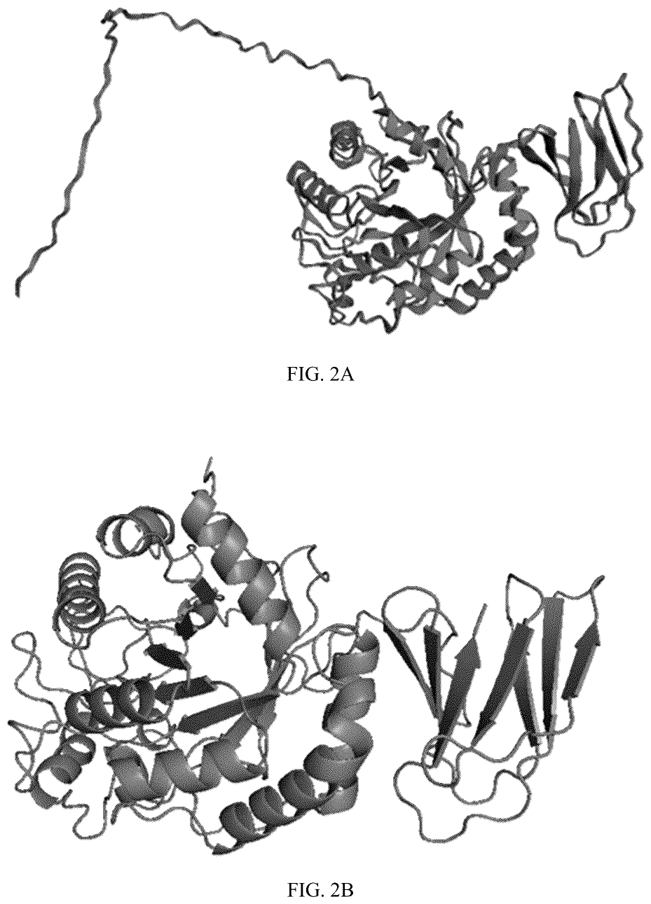

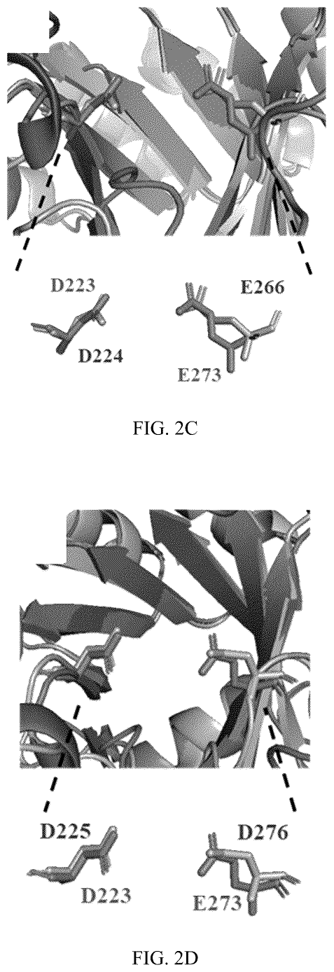

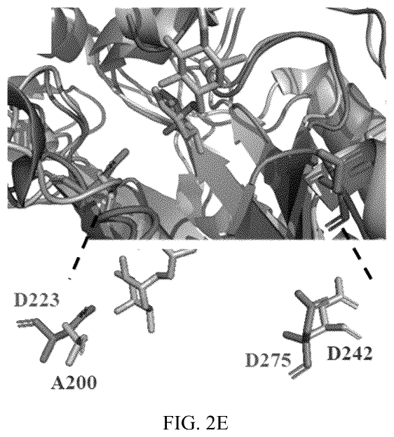

To investigate the possible acid/base residues based on a structural homology study with other characterized GH29A α-L-fucosidases, a theoretical 3D structural model of PnfucA was generated using AlphaFold 2. As shown in FIGS. 2A and 2B, the projected structure showed a classic GH29 (β/α)₈ barrel structure connected to a C-terminal β-sandwich domain with a high degree of reliability, as indicated by a high per-residue confidence metric (pLDDT; predicted local distance difference test) of over 90. The N-terminal region of the model displayed a very low confidence score (pLDDT <50). Such N-terminal region comprises signal peptide residues 1-20, and, due to poor prediction quality, this region was removed from the model.

A structure superposition analysis verified that the catalytic amino acid residues in PnfucA were E273 (general acid/base) and D242 (nucleophile) (FIGS. 2C and 2D). These residues are aligned similarly to those in TM aFuc and human α-L-fucosidase (FucA1), but they are different from those in AlfC (FIG. 2E). It was also to note that GH29 enzymes function as retaining hydrolases, using the classic Koshland double-displacement catalytic reaction^{[22]}.

### Example 2: Heterologous Expression of α-L-Fucosidases

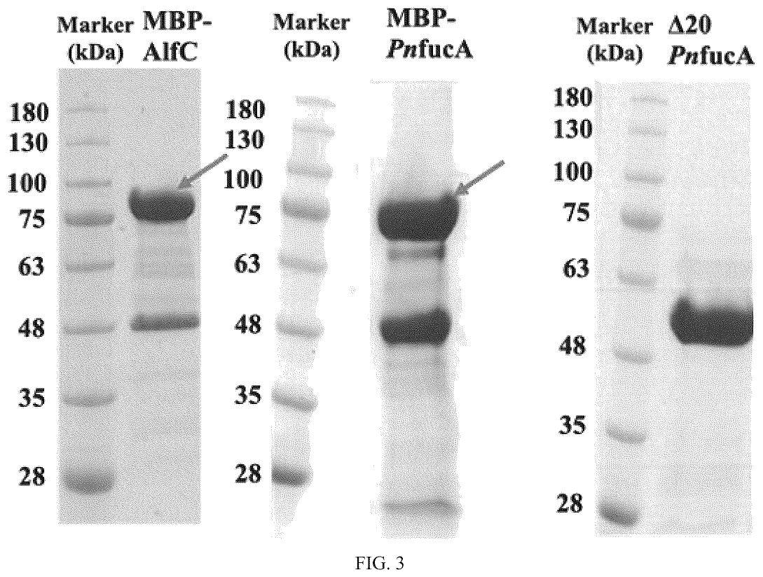

Recognizing PnfucA as a potential GH29A, the recombinant PnfucA was produced in E. coli cells. Also, the recombinant L. casei fucosidase AlfC was produced as a positive control. To improve protein solubility, the N-terminal maltose binding protein (MBP) tag was introduced into the pMAL-c4X vector which effectively enabled the expression of both PnfucA and AlfC in a soluble form (FIG. 3).

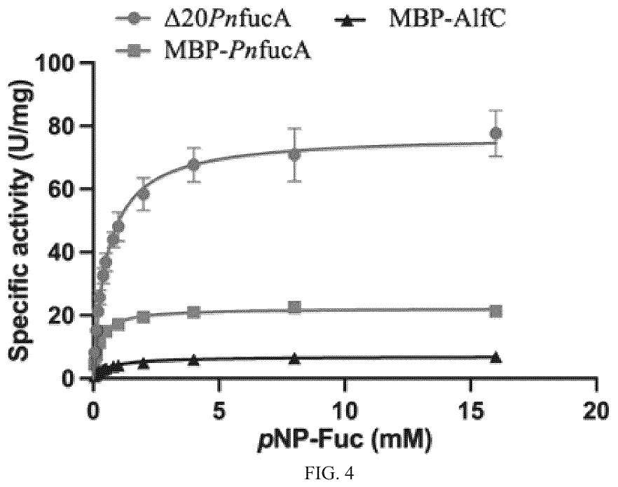

The MBP-PnfucA preparation showed remarkable catalytic efficiency (FIG. 4). To further enhance the yield and purity of MBP-fusion PnfucA for wider and more versatile applications, the pET-16b vector with first twenty amino acids removed from the N-terminal region was adopted, leading to the successful production of PnfucA with an apparent molecular weight of about 52 kDa (FIG. 3), referred to as Δ20PnfucA. After affinity purification, the yield of Δ20PnfucA increased by approximately 1,000-fold, achieving over 30 mg/L with outstanding purity. This substantial yield of Δ20PnfucA enabled the enzyme's biochemical characterization.

### Example 3: Biochemical Characterization of Heterologous Expressed PnfucA Enzyme

In assessing the biochemical characteristics of recombinant Δ20PnfucA, the synthetic substrate p-nitrophenyl-α-L-fucopyranoside (pNP-Fuc) was used as the substrate of choice. On enzymatic hydrolysis, the pNP-Fuc undergoes cleavage to release p-nitrophenyl (pNP).

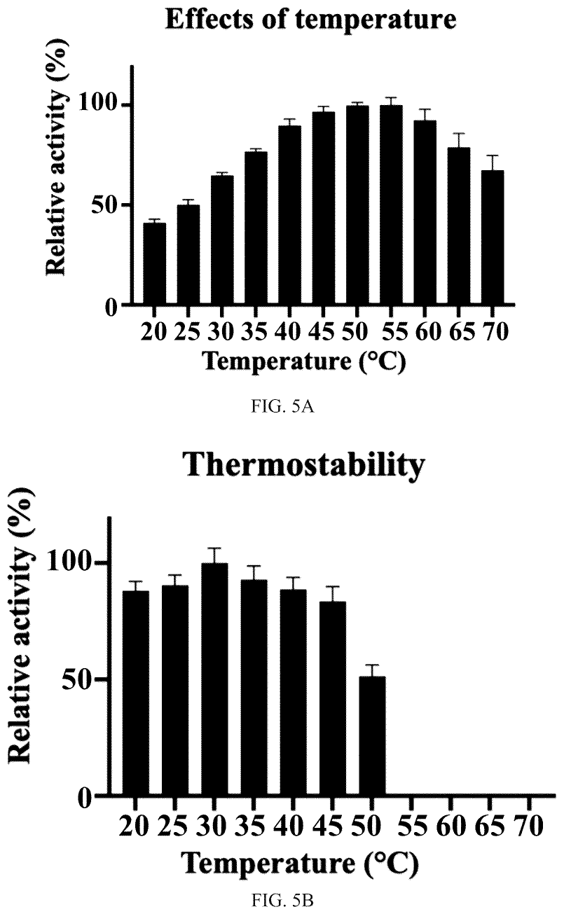

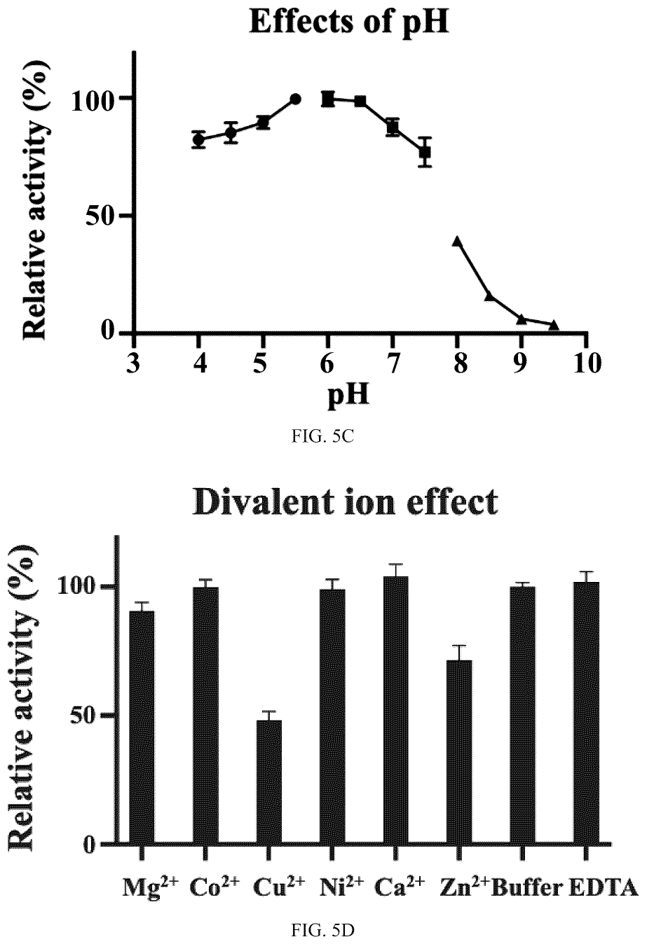

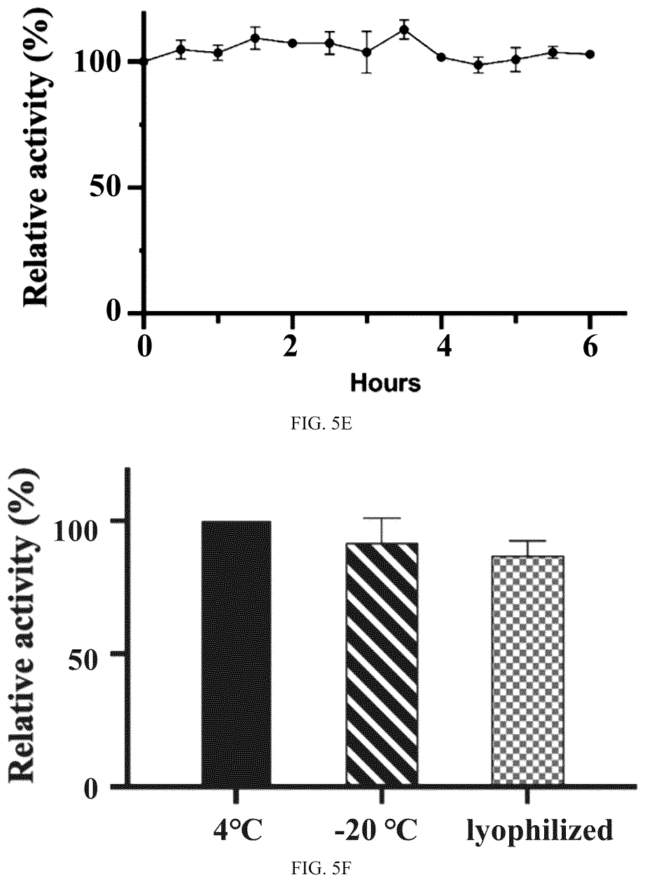

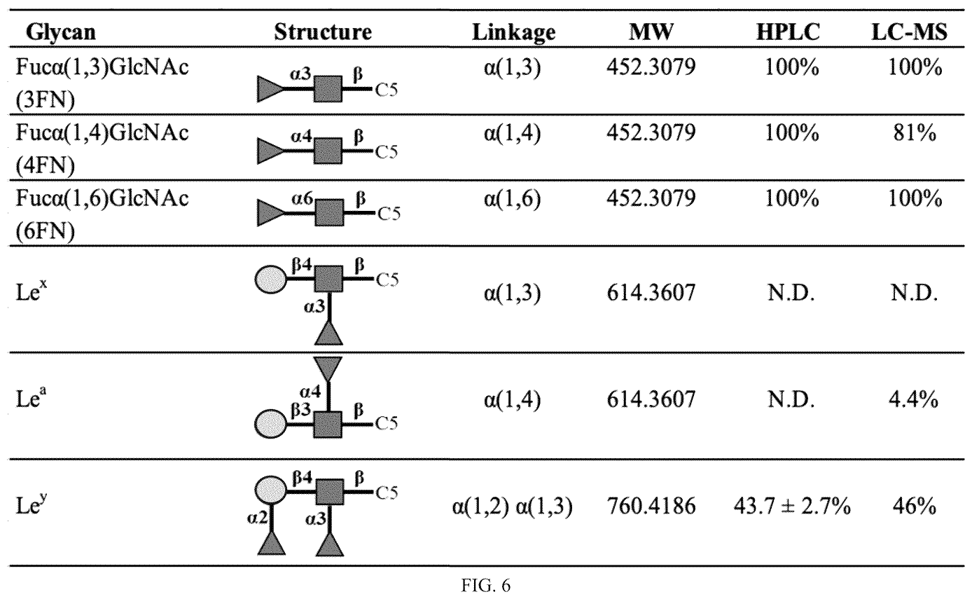

As shown in FIGS. 5A and 5B, the Δ20PnfucA exhibited enzymatic activity at temperatures between 50 °C and 55 °C, and the enzyme retained over 80% of its activity at temperatures below 45 °C. The enzymatic activity of Δ20PnfucA was observed at slightly acidic pH of 5.5 to 6.5, and the presence of copper (Cu²⁺) and zinc (Zn²⁺) ions was detrimental to its activity (FIGS. 5C and 5D). Full activity was maintained even after incubation (at pH 7) for 6 hours at 37 °C (FIG. 5E).

To further assess the stability of Δ20PnfucA, the enzyme was stored at −20 °C, followed by freeze drying. After 28 days of storage, over 92±9% and 87±6% of the enzyme activity was retained at −20 °C and in the freeze-dried state, respectively (FIG. 5F).

The kinetics parameters k_{cat} and Kₘ for Δ20PnfucA were calculated at 66.5±2 s⁻¹ and 0.56±0.05 mM, respectively. The Δ20PnfucA exhibited a significantly higher Vₘₐₓ (77.2±0.1 U/mg) compared with the MBP-fusion PnfucA (22.2±0.4 U/mg) and MBP-fusion AlfC (7.0±0.2 U/mg), as shown in FIG. 4. This observation indicated that fusion with MBP negatively impacts PnfucA activity.

### Example 4: Analysis of the Substrate Specificity of PnfucA

Although pNP-Fuc is a commonly used substrate for the enzymatic characterization of GH29A α-L-fucosidases, GH29B enzymes do not hydrolyze this synthetic substrate^{[23]}. Furthermore, hydrolysis of pNP-Fuc does not elucidate the enzyme's regiospecificity for α(1,2)-, α(1,3)-, α(1,4)-, and α(1,6)-linked substrates. For instance, both Bacteroides thetaiotaomicron BT-2970 and Elizabethkingia meningoseptica cFase are active with pNP-Fuc, but are inactive with the Fucα(1,6) GlcNAc disaccharide. Therefore, to facilitate a meaningful kinetic comparison among characterized α-L-fucosidases, further investigation using disaccharide substrates was conducted in this example.

The substrate specificity of Δ20PnfucA was determined by incubating the enzyme in a range of synthetic glycans (di-, tri-, and oligosaccharides) containing fucose residue(s) and the reducing end C-5 amino linker. The presence of C-5 amino linker did not alter the structure of glycan substrates and was employed for subsequent conjugation with Cy5 mono NHS Ester. After reacting for 16 hours, the products were analyzed using both high-performance liquid chromatography (HPLC) and liquid chromatography-mass spectrometry (LC-MS).

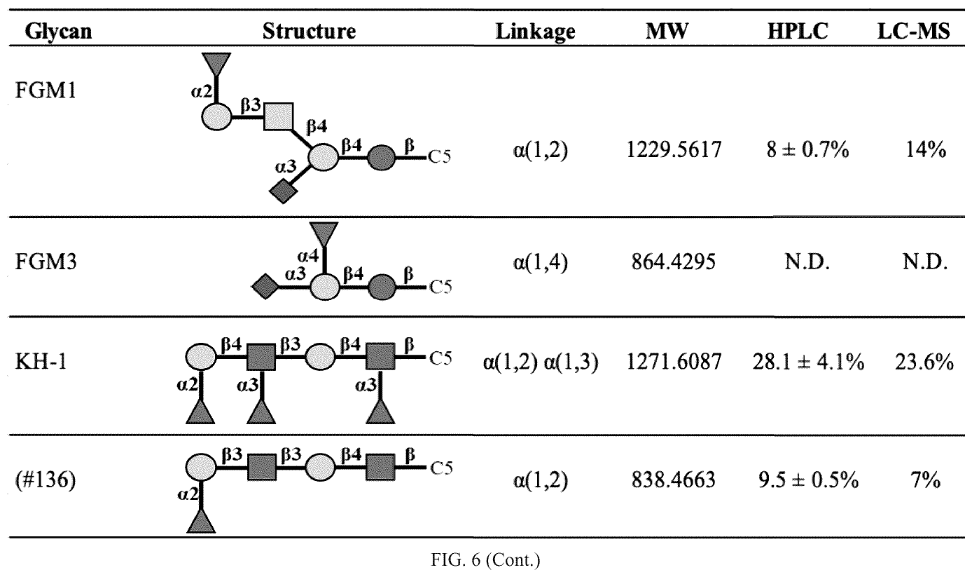

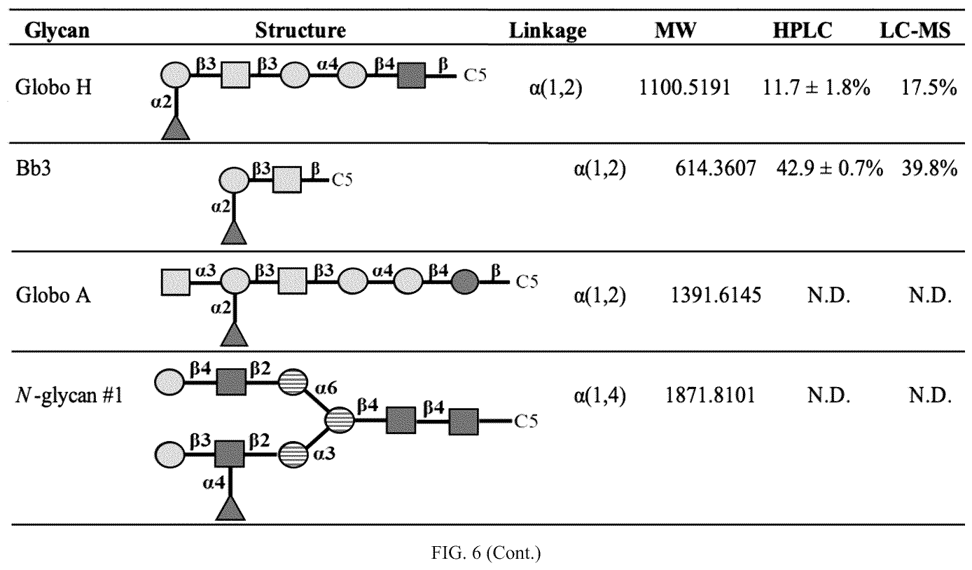

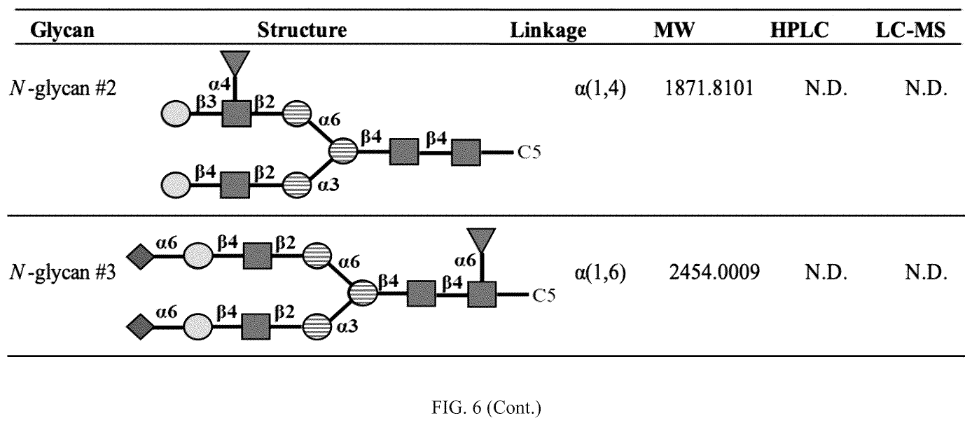

The results were reported in FIG. 6. With the substrates having core fucosylated disaccharides, PnfucA showed the superior abilities to liberate fucose from Fucα(1,3) GleNAc with C-5 amino linker (3FN-C5), Fucα(1,4) GlcNAc with C-5 amino linker (4FN-C5), and Fucα(1,6) GlcNAc with C-5 amino linker (6FN-C5). The HPLC analysis showed that the substrates were completely hydrolyzed. Furthermore, the LC-MS analysis showed that the extent of fucose removal surpassed 95% for both 3FN-C5 and 6FN-C5, highlighting the enzyme's exceptional effectiveness in cleaving this substrate category. In addition, there was no evidence of hydrolytic activity against the C-5 linked Lewis antigen x (Le^{x}), Lewis antigen a (Le^{a}), fucosylated GM3 ganglioside 3 (FGM3), Globo A, and sub-terminal α(1,4)-fucosylated or α(1,6)-fucosylated disialyl-N-glycans. These results clearly demonstrated that PnfucA had no activity on subterminal fucosylated oligosaccharides. Further, there was a moderate hydrolytic activity detected on C-5 linked fucosylated GM1 ganglioside 1 (FGM1), synthetic oligosaccharide #136 (pentasaccharide with a terminal (1,2)-linked fucose), Globo H, and Bb3 glycans. Nevertheless, only 8% to 42.9% of terminal fucose was released from these substrates with higher degrees of polymerization (DPs). These results implied a preference of PnfucA for core FN. With di- and tri-fucosylated substrates, such as Lewis antigen y (Le^{y}) and antigen KH-1, hydrolysis removed a single fucose residue (−146 Da), demonstrating the ability of PnfucA to target and remove terminal fucose residues.

The kinetic parameters and the substrate preferences of some GH29A α-L-fucosidases had been summarized in Tables 1 and 2 below.

TABLE 1
Kinetic parameters of different GH29A α-L-fucosidases with
pNP-fucose as the substrate

| | | | k_{cat}/Kₘ | Sequence |
|---|---|---|---|---|
| Organism | k_{cat} (s⁻¹) | Kₘ (mM) | (s⁻¹ mM⁻¹) | identity* |
| Thermotoga | 5.4 ± 0.2 | 0.03 ± 0.00 | 158.8 | 21.7% |
| maritima | | | | |
| L. casei AlfC | 1.38 ± 0.02 | 0.70 ± 0.03 | 2.0 | 18.6% |
| Bacteroides | 183.8 | 0.44 | 420.6 | 53.1% |
| fragilis BfFucH | | | | |
| Bacteroides | 1.3 | 1.5 | 0.87 | 22.3% |
| thetaiotaomicron | | | | |
| BT-2970 | | | | |
| Elizabethkingia | 0.14 | 0.60 | 0.23 | 23.1% |
| anophelis cFase | | | | |
| P. nigrescens | 66.5 ± 2 | 0.56 ± 0.05 | 118.8 | — |
| A20PnfucA | | | | |
| P. nigrescens | 35.4 ± 0.64 | 0.25 ± 0.02 | 141.6 | — |
| MBP-PnfucA | | | | |
| L. casei MBP-AlfC | 9.7 ± 0.28 | 0.62 ± 0.06 | 15.6 | — |

*Sequence identity relative to PnfucA

TABLE 2
Substrate specificity of some GH29A α-L-
fucosidases presented in the phylogenetic tree

| | Substrate | | | | | | |
|---|---|---|---|---|---|---|---|
| | | | | | Fucα(1,3) | Fucα(1,4) | Fucα(1,6) |
| Enzyme | 2′-FL | 3-FL | Le^{x} | Le^{a} | GleNAc | GlcNAc | GlcNAc |
| AlfC | − | − | − | − | + | + | + |
| BT-2970 | Trace | − | + | + | − | − | − |
| FgFCO1 | + | − | x | − | x | x | x |
| TfFuc1 | + | − | − | − | + | + | + |
| Mfuc5 | + | + | − | + | − | + | + |
| cFase I | − | + | + | − | x | x | − |
| NkFuc | − | − | − | − | − | − | + |
| BfFucH | x | x | − | − | x | x | + |
| PnfucA | x | x | − | − | + | + | + |

2′-FL: 2′-fucosyllactose; 3-FL: 3-fucosyllactose; x: not tested

These results indicated that Δ20PnfucA showed broad linkage specificity with a marked preference for disaccharides substrates, whereas BT-2970 hydrolyzed only the sub-terminal fucosylated substrates Le^{x} and Le^{y} but not core FN.

### Example 5: Defucosylation of N-Linked Glycoprotein Using PnFucA

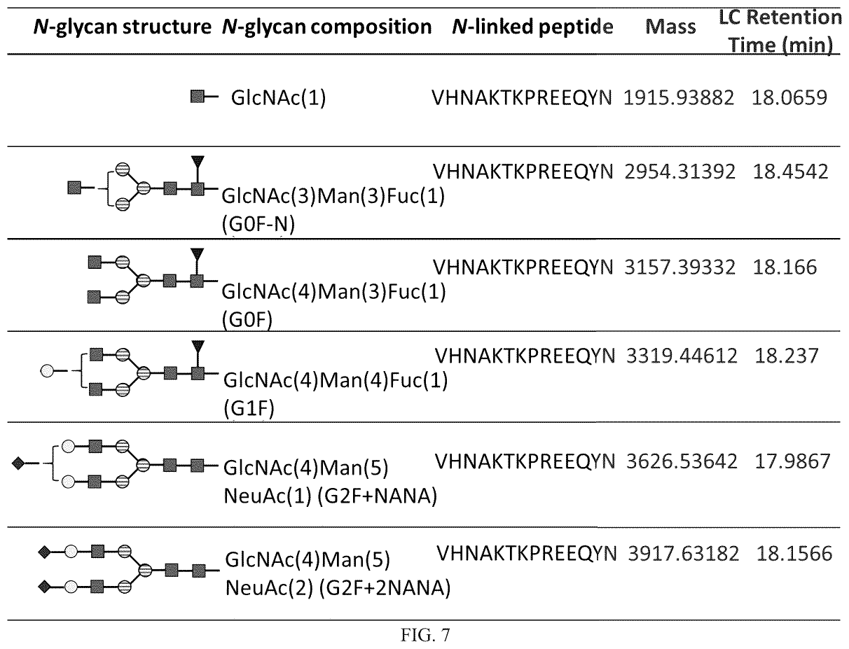

Prior to conducting the defucosylation reactions on the model N-linked glycoprotein mAb adalimumab (Humira), the structural profiles of N-glycans on Asn 297 in the Fc segment of adalimumab were analyzed. Consistent with the literature^{[24]}, the LC-ESI-MS analysis revealed that all the N-glycans were fucosylated, with a distribution of 2.4% mono-N-acetylglucosaminylated (GOF-N), 84.7% agalactosylated (GOF), and 12.9% mono-galactosylated (G1F) complex-type N-glycans (FIG. 7).

The extent of defucosylation of adalimumab by A20Pnfuc was monitored using Western blot analysis with the fucose-specific Aleuria aurantia lectin (AAL). The presence of α(1,6)-linked Fucp was confirmed by positive AAL staining in both untreated adalimumab and Endo-S2 treated adalimumab. Furthermore, when adalimumab was treated only with Δ20PnfucA, strong AAL staining was observed, indicating the enzyme failed to remove the core fucose in extended N-glycans. It was also consistent with the failure of Δ20PnfucA to hydrolyze the fucose residues on C-5 linked α(1,6)-core-fucosylated sialyl-complex-type N-glycan (N-glycan #3, FIG. 6).

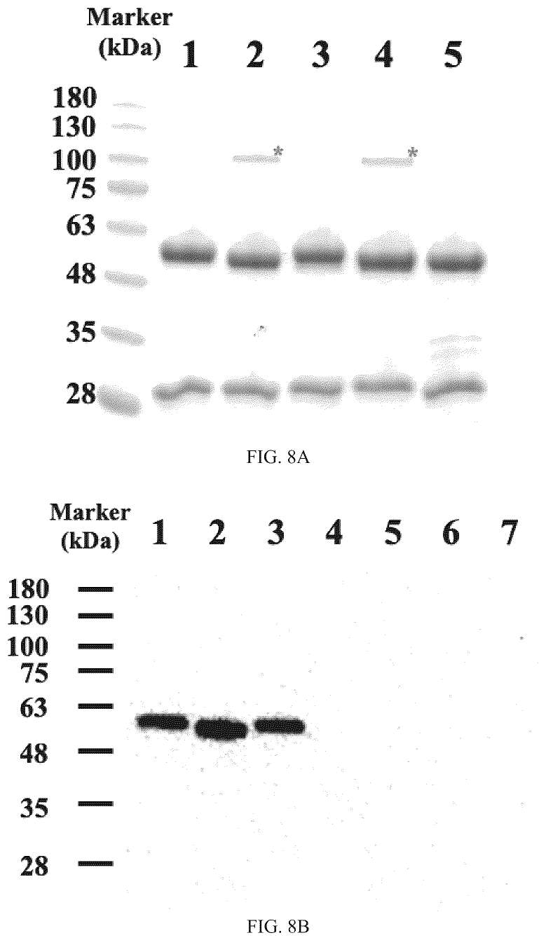

Following the treatment of adalimumab with Endo-S2 to give adalimumab containing a core 6FN disaccharide (Hum_{Fucα(1,6)-GlcNAc}), defucosylation was carried out with Δ20PnfucA, leading to the complete removal of core fucose from Hum_{Fucα(1,6)}-GlcNAc, with a specific activity of 9.2±1.3 μg per μg of Δ20PnfucA per hour. These results suggested that PnfucA selectively targeted terminal fucose residues, such as those in core FN (FIGS. 8A and 8B).

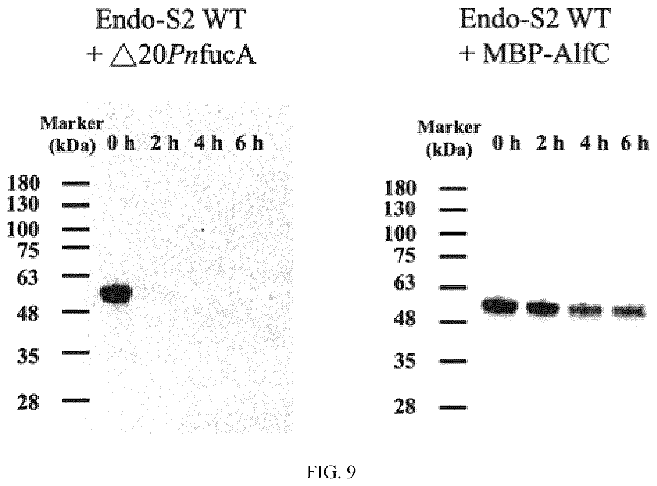

A comparative study was undertaken using MBP-fusion α-L-fucosidases. Using Δ20PnfucA, defucosylation was fast, with MBP-PnfucA achieving complete defucosylation within 2 hours, whereas MBP-AlfC showed a slower defucosylation rate, with residual core-α(1,6) fucose still present after 6 hours (FIG. 9).

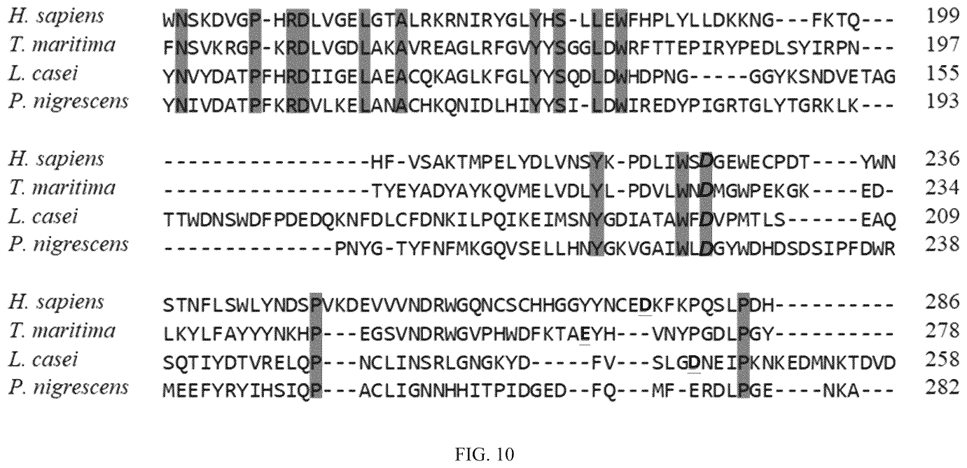

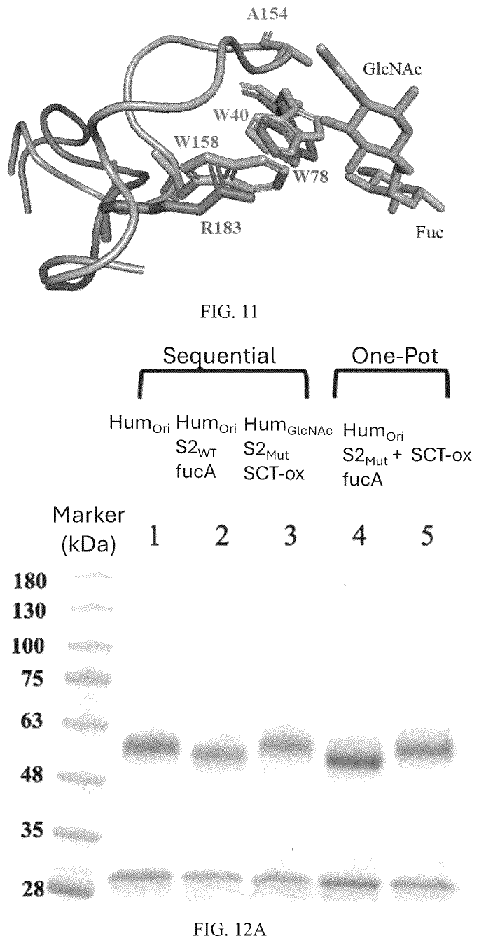

Sequence alignment analysis showed a notable difference between PnfucA and AlfC. The targeting of Fucα(1,6) GlcNAc by AlfC was attributed to the GlcNAc residue within the hydrophobic pocket formed by Trp 40, Ala 154, and Trp 158. Specifically, the Ala 154 and the Trp 158 are situated within a loop consisting of 19 amino acids (ranging from Gly 145 to Asp 163), whereas PnfucA has a much shorter loop with only 10 amino acids (from Ile 181 to Arg 190) as shown in FIG. 10. The absence of the homologous hydrophobic pocket in PnfucA may have an impact on the substrate binding of these two enzymes, as shown in FIG. 11.

### Example 6: One-Pot Two-Step Fc Domain Glycoengineering Using PnfucA

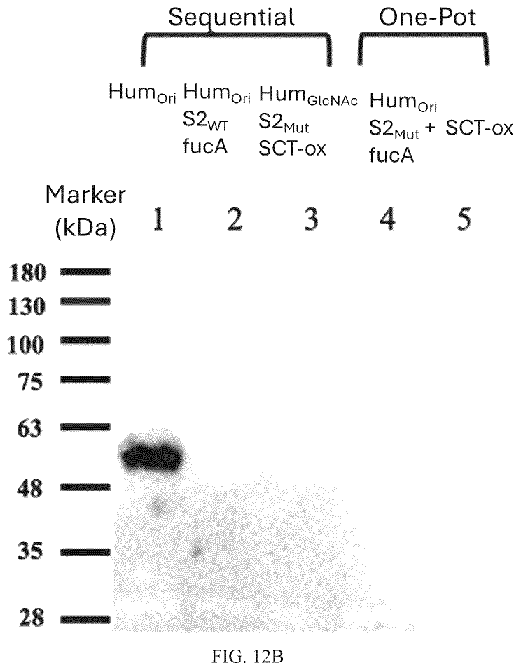

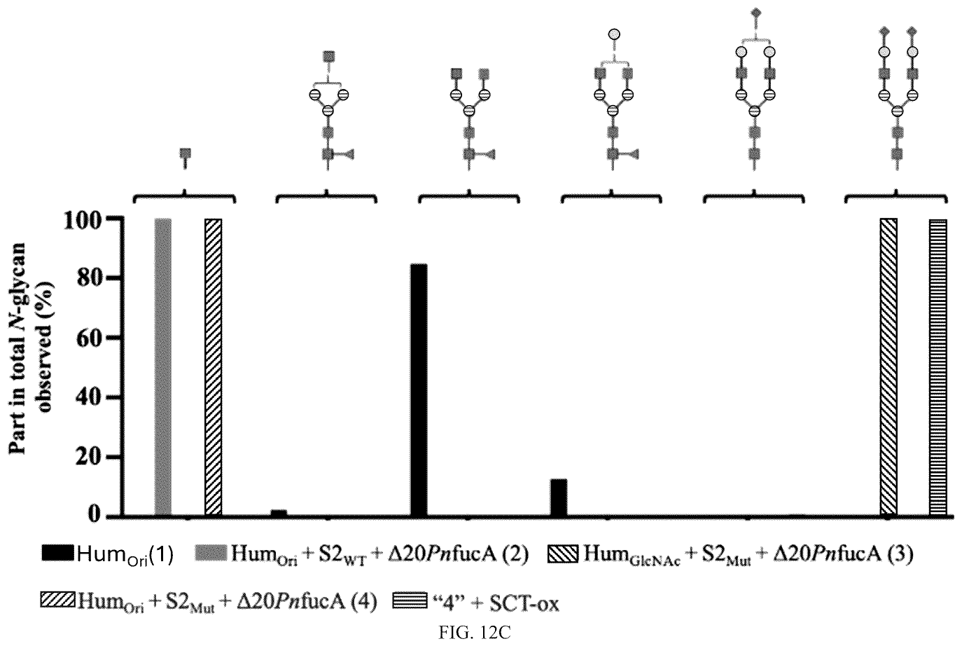

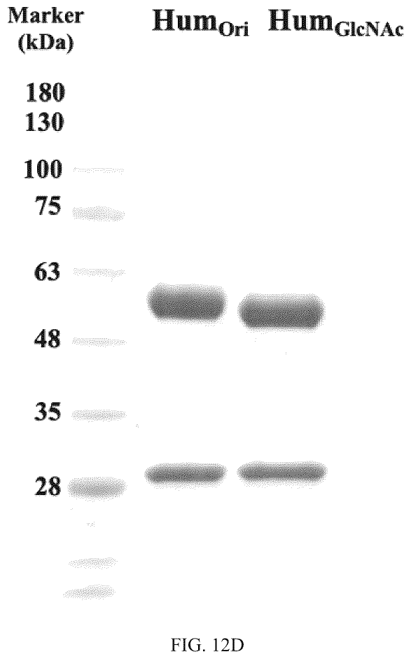

The currently used method for glycoengineering mAbs to enhance effector functions involves a three-step process using three enzymes: an endo-β-N-acetylglucosaminidase and its mutant glycosynthase, together with a GH29 α-L-fucosidase. As an example, adalimumab (Humira) was used to produce a uniform α(2,6)-sialylated biantennary complex-type (SCT) N-glycan. The process was initiated with Endo-S2, which removed heterogeneous Fc N-glycans. Subsequently, Δ20PnfucA was used to remove the core-α(1,6) fucose residue, resulting in adalimumab with the first GlcNAc bridgehead as the acceptor (Hum_{GlcNAc}) (FIGS. 12A and 12B, lane 1). The composition of the Fc N-glycans was shown in FIG. 12C, and the corresponding mass values for each glycosylated peptide from thermolysin-digested adalimumab could be seen in FIG. 7.

After purifying Hum_{GlcNAc} (FIG. 12D), transglycosylation was carried out using the Endo-S2 mutant D184M, with SCT-oxazoline (SCT-ox) as the sugar donor, yielding glycoengineered adalimumab with a uniform SCT N-glycan (Hum_{SCT}) (FIGS. 12A and 12B, lane 3) in quantitative yield within 1 hour. It was observed that Endo-S2 mutant D184M could also perform hydrolysis effectively on Fc N-glycans of adalimumab, achieving a rate of 54.6±13.7 μg per μg of enzyme per hour, despite it being previously reported that this mutant enzyme showed only very low hydrolytic activity (about 1.4% compared with the wild-type enzyme) (FIGS. 12A and 12B, lane 4). Additionally, the transglycosylation rate was found to be 74.5±2.9 μg of adalimumab per μg of Endo-S2 mutant D184M per hour.

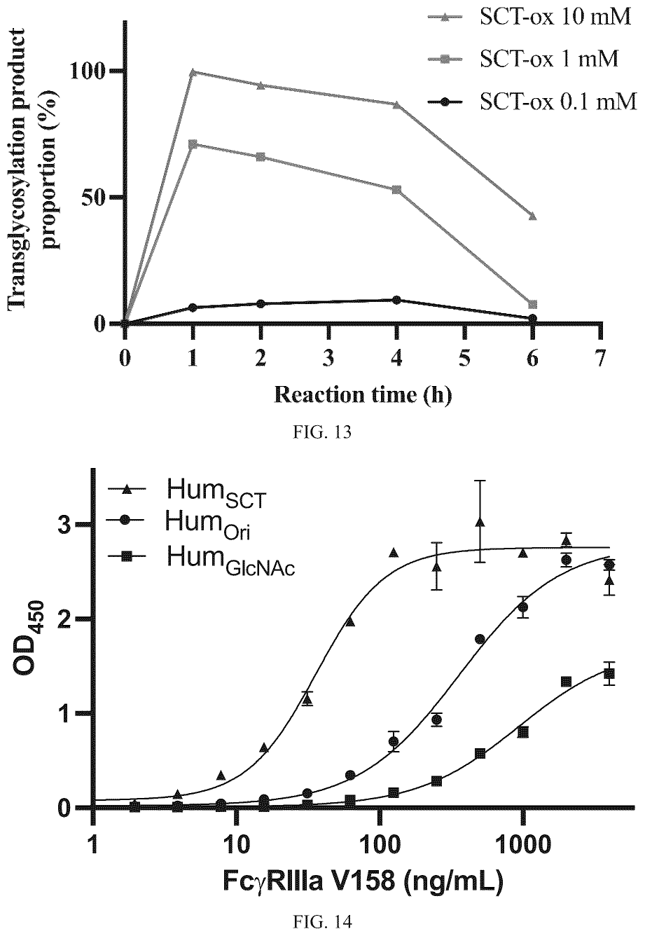

Further, the SCT-ox concentration for the transglycosylation reaction was then evaluated and optimized (FIG. 13). After a 1-hour reaction, the conversions to the transglycosylated product Hum_{SCT} were 100%, 71%, and 6% in the presence of 10 mM, 1 mM, and 0.1 mM SCT-ox, respectively. The proportion of Hum_{SCT} decreased from the second hour and dropped abruptly after the fourth hour to 8% for 1 mM SCT-ox and 43% for 10 mM SCT-ox. In contrast, for 0.1 mM SCT-ox, it remained around 7% between the first and fourth hour and fell to 2% at the sixth hour.

Based on these results, a One-Pot Two-Step reaction was proposed to generate homogeneous Hum_{SCT} within 4 hours, consisting of 3 hours of deglycosylation, followed by 1 hour of transglycosylation. In the first step, the antibody adalimumab was defucosylated in the presence of Δ20PnfucA and the Endo-S2 mutant. The reaction was successfully completed within 3 hours to afford Hum_{GlcNAc} with a quantitative yield, as shown in FIGS. 12A and 12B, lane 4. In the subsequent step, 10 mM SCT-ox was introduced into the same reaction vessel, and the reaction was allowed to proceed for 1 hour, resulting in the formation of the transglycosylated product Hum_{SCT} with a 100% yield (FIGS. 12A and 12B, lane 5).

These results indicated that Δ20PnfucA with only the mutant Endo-S2 D184M in a One-Pot method may provide an effective and straightforward strategy for glycoengineering. This method streamlined the transformation of a heavily fucosylated monoclonal antibody, synthesized in Chinese hamster ovary (CHO) cells, into a uniformly defucosylated glycoform within 4 hours, eliminating the need for extra purification steps. With Hum_{GlcNAc} and Hum_{SCT} in hand, obtained from the One-Pot reaction (FIG. 14), it was observed that the binding affinities of Hum_{SCT} to FcγRIIIa V158 were higher (EC50=35.9 ng/ml) compared to those of Hum_{Ori} (EC50=358.4 ng/mL) and Hum_{GlcNAc} (EC50=928.6 ng/ml). This method also implied enhanced ADCC activity, showcasing the potential applications of an improved antibody glycoengineering strategy.

From the above, it can be clearly seen that the fucosidase provided herein enables the efficient and seamless integration of biantennary glycans in a one-pot fashion, resulting in an antibody with a homogeneous afucosylated glycoform that exhibits an improved binding affinity toward Fcγ receptor IIIa. Therefore, the fucosidase and the method provided herein facilitate the optimization of Fc effector functions and are useful for improving the therapeutic outcomes.

It is obvious to a person skilled in the art that with the advancement of technology, the basic idea may be implemented in various ways. The embodiments are thus not limited to the examples described above; instead, they may vary within the scope of the claims.

The embodiments described hereinbefore may be used in any combination with each other. Several of the embodiments may be combined together to form a further embodiment. A compound, a composition, or a method disclosed herein may comprise at least one of the embodiments described hereinbefore. It will be understood that the benefits and advantages described above may relate to one embodiment or may relate to several embodiments. The embodiments are not limited to those that solve any or all of the stated problems or those that have any or all of the stated benefits and advantages.

### REFERENCES

[1] Shields, R. L.; Lai, J.; Keck, R.; O'Connell, L. Y.; Hong, K.; Meng, Y. G.; Weikert, S. H. A.; Presta, L. G., Lack of fucose on human IgG1 N-linked oligosaccharide improves binding to human FcγRIII and antibody-dependent cellular toxicity. Journal of Biological Chemistry. 2002, 277 (30), 26733-26740.
[2] Liu, C. P.; Tsai, T. I.; Cheng, T.; Shivatare, V. S.; Wu, C. Y.; Wu, C. Y.; Wong, C. H., Glycoengineering of antibody (Herceptin) through yeast expression and in vitro enzymatic glycosylation. Proceedings of the National Academy of Sciences USA. 2018, 115 (4), 720-725.
[3] Junttila, T. T.; Parsons, K.; Olsson, C.; Lu, Y.; Xin, Y.; Theriault, J.; Crocker, L.; Pabonan, O.; Baginski, T.; Meng, G.; Totpal, K.; Kelley, R. F.; Sliwkowski, M. X., Superior in vivo efficacy of afucosylated trastuzumab in the treatment of HER2-amplified breast cancer. Cancer Research. 2010, 70 (11), 4481-4489.
[4] Li, T.; DiLillo, D. J.; Bournazos, S.; Giddens, J. P.; Ravetch, J. V.; Wang, L.-X., Modulating IgG effector function by Fc glycan engineering. Proceedings of the National Academy of Sciences USA. 2017, 114 (13), 3485-3490.
[5] Kommineni, V.; Markert, M.; Ren, Z.; Palle, S.; Carrillo, B.; Deng, J.; Tejeda, A.; Nandi, S.; McDonald, K. A.; Marcel, S.; Holtz, B. In vivo glycan engineering via the mannosidase I inhibitor (kifunensine) improves efficacy of rituximab manufactured in Nicotiana benthamiana plants. International Journal of Molecular Sciences. 2019, 20 (1): 194.
[6] Rillahan, C. D.; Antonopoulos, A.; Lefort, C. T.; Sonon, R.; Azadi, P.; Ley, K.; Dell, A.; Haslam, S. M.; Paulson, J. C., Global metabolic inhibitors of sialyl- and fucosyltransferases remodel the glycome. Nature Chemical Biology. 2012, 8 (7), 661-668.
[7] Ehret, J.; Zimmermann, M.; Eichhorn, T.; Zimmer, A., Impact of cell culture media additives on IgG glycosylation produced in Chinese hamster ovary cells. Biotechnology and Bioengineering. 2019, 116 (4), 816-830.
[8] Yamane-Ohnuki, N.; Kinoshita, S.; Inoue-Urakubo, M.; Kusunoki, M.; Iida, S.; Nakano, R.; Wakitani, M.; Niwa, R.; Sakurada, M.; Uchida, K.; Shitara, K.; Satoh, M., Establishment of FUT8 knockout Chinese hamster ovary cells: an ideal host cell line for producing completely defucosylated antibodies with enhanced antibody-dependent cellular cytotoxicity. Biotechnology and Bioengineering. 2004, 87 (5), 614-622.
[9] Malphettes, L.; Freyvert, Y.; Chang, J.; Liu, P.-Q.; Chan, E.; Miller, J. C.; Zhou, Z.; Nguyen, T.; Tsai, C.; Snowden, A. W.; Collingwood, T. N.; Gregory, P. D.; Cost, G. J., Highly efficient deletion of FUT8 in CHO cell lines using zinc-finger nucleases yields cells that produce completely nonfucosylated antibodies. Biotechnology and Bioengineering. 2010, 106 (5), 774-783.
[10] Yang, G.; Wang, Q.; Chen, L.; Betenbaugh, M. J.; Zhang, H., Glycoproteomic characterization of FUT8 knock-out CHO cells reveals roles of FUT8 in the glycosylation. Frontiers in Chemistry. 2021, 9, 755238.
[11] Niwa, R.; Shoji-Hosaka, E.; Sakurada, M.; Shinkawa, T.; Uchida, K.; Nakamura, K.; Matsushima, K.; Ueda, R.; Hanai, N.; Shitara, K., Defucosylated chimeric anti-CC chemokine receptor 4 IgG1 with enhanced antibody-dependent cellular cytotoxicity shows potent therapeutic activity to T-cell leukemia and lymphoma. Cancer Research. 2004, 64 (6), 2127-2133.
[12] Niwa, R.; Sakurada, M.; Kobayashi, Y.; Uehara, A.; Matsushima, K.; Ueda, R.; Nakamura, K.; Shitara, K., Enhanced natural killer cell binding and activation by low-fucose IgG1 antibody results in potent antibody-dependent cellular cytotoxicity induction at lower antigen density. Clinical Cancer Research. 2005, 11 (6), 2327-2336.
[13] Shinkawa, T.; Nakamura, K.; Yamane, N.; Shoji-Hosaka, E.; Kanda, Y.; Sakurada, M.; Uchida, K.; Anazawa, H.; Satoh, M.; Yamasaki, M.; Hanai, N.; Shitara, K., The absence of fucose but not the presence of galactose or bisecting N-acetylglucosamine of human IgG1 complex-type oligosaccharides shows the critical role of enhancing antibody-dependent cellular cytotoxicity. Journal of Biological Chemistry. 2003, 278 (5), 3466-3473.
[14] Rodriguez-Diaz, J.; Monedero, V.; Yebra, M. J., Utilization of natural fucosylated oligosaccharides by three novel α-L-fucosidases from a probiotic Lactobacillus casei strain. Applied and Environmental Microbiology. 2011, 77 (2), 703-705.
[15] Li, T.; Tong, X.; Yang, Q.; Giddens, J. P.; Wang, L.-X., Glycosynthase mutants of endoglycosidase S2 show potent transglycosylation activity and remarkably relaxed substrate specificity for antibody glycosylation remodeling. Journal of Biological Chemistry. 2016, 291 (32), 16508-16518.
[16] Tsai, T. I.; Li, S. T.; Liu, C. P.; Chen, K. Y.; Shivatare, S. S.; Lin, C. W.; Liao, S. F.; Lin, C. W.; Hsu, T. L.; Wu, Y. T.; Tsai, M. H.; Lai, M. Y.; Lin, N. H.; Wu, C. Y.; Wong, C. H., An effective bacterial fucosidase for glycoprotein remodeling. ACS Chemical Biology. 2017, 12 (1), 63-72.
[17] Bradford, M. M., A rapid and sensitive method for the quantitation of microgram quantities of protein utilizing the principle of protein-dye binding. Analytical Biochemistry. 1976, 72 (1), 248-254.
[18] Lee, H. Y.; Chen, C. Y.; Tsai, T. I.; Li, S. T.; Lin, K. H.; Cheng, Y. Y.; Ren, C. T.; Cheng, T. J. R.; Wu, C. Y.; Wong, C. H., Immunogenicity study of Globo H analogues with modification at the reducing or nonreducing end of the tumor antigen. Journal of the American Chemical Society. 2014 136 (48), 16844-16853.
[19] Shivatare, S. S.; Chang, S. H.; Tsai, T. I.; Tseng, S. Y.; Shivatare, V. S.; Lin, Y. S.; Cheng, Y. Y.; Ren, C. T.; Lee, C. C. D.; Pawar, S.; Tsai, C. S.; Shih, H. W.; Zeng, Y. F.; Liang, C. H.; Kwong, P. D.; Burton, D. R.; Wu, C. Y.; Wong, C. H., Modular synthesis of N-glycans and arrays for the hetero-ligand binding analysis of HIV antibodies. Nature Chemistry. 2016, 8 (4), 338-346.
[20] Tarling, C. A.; He, S.; Sulzenbacher, G.; Bignon, C.; Bourne, Y.; Henrissat, B.; Withers, S. G., Identification of the catalytic nucleophile of the family 29 α-L-fucosidase from Thermotoga maritima through trapping of a covalent glycosyl-enzyme intermediate and mutagenesis. Journal of Biological Chemistry. 2003, 278 (48), 47394-47399.
[21] Klontz, E. H.; Li, C.; Kihn, K.; Fields, J. K.; Beckett, D.; Snyder, G. A.; Wintrode, P. L.; Deredge, D.; Wang, L.-X.; Sundberg, E. J., Structure and dynamics of an α-fucosidase reveal a mechanism for highly efficient IgG transfucosylation. Nature Communications. 2020, 11 (1), 6204.
[22] Lammerts van Bueren, A.; Ardevol, A.; Fayers-Kerr, J.; Luo, B.; Zhang, Y.; Sollogoub, M.; Bleriot, Y.; Rovira, C.; Davies, G. J., Analysis of the reaction coordinate of α-L-fucosidases: a combined structure and quantum mechanical approach. Journal of the American Chemical Society. 2010, 132 (6), 1804-1806.
[23] Sakurama, H.; Tsutsumi, E.; Ashida, H.; Katayama, T.; Yamamoto, K.; Kumagai, H., Differences in the substrate specificities and active-site structures of two α-L-fucosidases (glycoside hydrolase family 29) from Bacteroides thetaiotaomicron. Bioscience, Biotechnology, and Biochemistry. 2012, 76 (5), 1022-1024.
[24] Tebbey, P. W.; Varga, A.; Naill, M.; Clewell, J.; Venema, J., Consistency of quality attributes for the glycosylated monoclonal antibody Humira (adalimumab). mAbs. 2015, 7 (5), 805-811.

What is claimed is:

1. A composition for defucosylating an N-glycan, comprising an α-L-fucosidase which has an amino acid sequence having at least 80% identical to SEQ ID NO: 1 and has an enzymatic activity for hydrolyzing an α(1,6)-linked core fucose present in the N-glycan.

2. The composition of claim 1, wherein the α-L-fucosidase is a truncated form of SEQ ID NO: 1.

3. The composition of claim 2, wherein the truncated form of SEQ ID NO: 1 has amino acids 21 to 452 of SEQ ID NO: 1.

4. The composition of claim 1, further comprising a glycosidase or a glycosynthase, wherein the glycosidase is selected from the group consisting of endo-β-N-acetylglucosaminidase, galactosidase, neuraminidase, mannosidase, and any combination thereof.

5. The composition of claim 4, wherein the glycosidase or the glycosynthase is selected from the group consisting of EndoA, EndoCC, EndoF1, EndoF2, EndoF3, EndoH, EndoM, EndoS, EndoS2, and a variant thereof.

6. The composition of claim 1, wherein the α-L-fucosidase is present in a lyophilized form.

7. A method for defucosylating an N-glycan, the method comprising:
providing an α-L-fucosidase and a glycoconjugate including the N-glycan having an α(1,6)-linked core fucose, wherein the α-L-fucosidase has an amino acid sequence having at least 80% identical to SEQ ID NO: 1 and has an enzymatic activity for hydrolyzing the α(1,6)-linked core fucose present in the N-glycan; and
incubating the α-L-fucosidase with the glycoconjugate under a condition for cleaving the α(1,6)-linked core fucose from the N-glycan.

8. The method of claim 7, further comprising incubating the glycoconjugate with an endo-β-N-acetylglucosaminidase and deglycosylating the glycoconjugate to produce a GlcNAc acceptor retaining the α(1,6)-linked core fucose.

9. The method of claim 8, further comprising cleaving the α(1,6)-linked core fucose from the GlcNAc acceptor by the α-L-fucosidase.

10. The method of claim 8, wherein the glycoconjugate is incubated simultaneously with the α-L-fucosidase and the endo-β-N-acetylglucosaminidase.

11. The method of claim 7, wherein the incubation is performed at about 35 °C to about 65 °C.

12. The method of claim 7, wherein the incubation is performed at a pH value in a range from 4.0 to 7.0.

13. The method of claim 7, wherein the glycoconjugate is a glycolipid, a glycoprotein, an oligosaccharide, or a glycopeptide.

14. The method of claim 13, wherein the glycoconjugate is a therapeutic glycoprotein selected from the group consisting of erythropoietin, a blood clotting factor, influenza hemagglutinin, an interferon, a colony-stimulating factor, a cytokine, an antibody, a Fc-fusion protein, an enzyme, and a hormone.

15. The method of claim 14, wherein the glycoprotein is a monoclonal antibody.

16. A method of producing an afucosylated glycoconjugate, the method comprising:
deglycosylating a glycoconjugate to produce a GlcNAc acceptor;
removing an α(1,6)-linked core fucose by exposing the GlcNAc acceptor to an α-L-fucosidase under a condition for defucosylating the GlcNAc-acceptor to obtain an afucosylated GlcNAc acceptor, wherein the α-L-fucosidase has an amino acid sequence having at least 80% identical to SEQ ID NO: 1 and has an enzymatic activity for hydrolyzing the α(1,6)-linked core fucose; and
enzymatically reacting the afucosylated GlcNAc acceptor with a sugar donor to produce the afucosylated glycoconjugate.

17. The method of claim 16, wherein the deglycosylation is carried out by endo-β-N-acetylglucosaminidase or a variant thereof.

18. The method of claim 16, wherein the enzymatical reaction of the afucosylated GlcNAc acceptor with the sugar donor is carried out by an endoglycosidase variant.

19. The method of claim 16, which is performed in a one-pot manner.

20. The method of claim 16, wherein the sugar donor is a synthetic oligosaccharide oxazoline or a sialylated oxazoline.


SEQUENCE LISTING (1 sequence)

SEQ ID NO: 1 - protein, 452 aa, Prevotella nigrescens
MKRIITTSLLGIALAIPIFAQTNSQQQESNNRATTTESVPQKYIPTAENLAARKRFEGFRFGIFLHWGIYSTFAQGEWYLNNKINKDEYAKAASAFYPSYFDADAWISAIKASGAKYITFTSRHHDSFSMFKTKESNYNIVDATPFKRDVLKELANACHKQNIDLHIYYSILDWIREDYPIGRTGLYTGRKLKPNYGTYFNFMKGQVSELLHNYGKVGAIWLDGYWDHDSDSIPFDWRMEEFYRYIHSIQPACLIGNNHHITPIDGEDFQMFERDLPGENKAGLSGQAISRLPLEACQTMNGMWGYKVSDINYKSTSQLIELLVRSAAKGSNLLLNIGPQPNGELPALALDRLQGIGKWMQQYGETIYETMAGDVAEADWGVSTSKGKKTYLHVLEQDAKTISCALSVKPKRVIDFASRQPLSFHYDKKTKTLTTHIGSHRNVVDYIIEIEK